(19) United States
(12) Patent Application Publication
(10) Pub. No.: US 2014/0051664 A1
(43) Pub. Date: Feb. 20, 2014

(54) COMBINATION THERAPY OF HSP90 INHIBITORY COMPOUNDS WITH RADIOTHERAPY

(71) Applicants: Kevin P. Foley, Landsdale, PA (US); David Proia, Newton, MA (US)

(72) Inventors: Kevin P. Foley, Landsdale, PA (US); David Proia, Newton, MA (US)

(73) Assignee: SYNTA PHARMACEUTICALS CORP., Lexington, MA (US)

(21) Appl. No.: 14/001,039

(22) PCT Filed: Feb. 22, 2012
(86) PCT No.: PCT/US12/26101

§ 371 Date: Oct. 31, 2013

Related U.S. Application Data
(60) Provisional application No. 61/445,589, filed on Feb. 23, 2011.

(51) Int. Cl. A61K 31/675; A61K 45/06; A61K 31/4196

(52) U.S. Cl. CPC A61K 31/675; A61K 31/4196; A61K 45/06
USPC 514/80; 514/384; 435/173.1

(57) ABSTRACT

Methods for treating cancer in a subject, comprising administering to the subject an effective amount of a compound represented by the following structural formula a tautomer, or a pharmaceutically acceptable salt thereof in combination with radiotherapy. The variables depicted in the structural formula are defined herein.

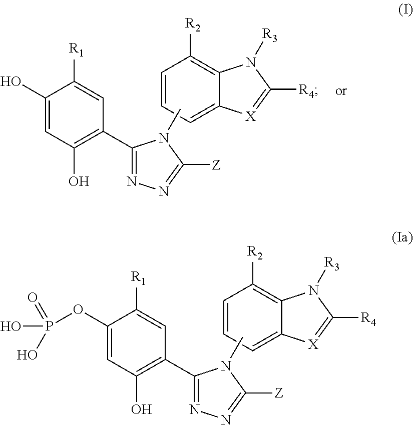

### CROSS-REFERENCE TO RELATED PATENT APPLICATIONS

This application claims the benefit of priority to U.S. Provisional Patent Application No. 61/445,589, filed on Feb. 23, 2011. The content of the above referenced application is incorporated herein by reference in its entirety.

### BACKGROUND OF THE INVENTION

Heat shock proteins (HSPs) are a class of chaperone proteins that are up-regulated in response to elevated temperature and other environmental stresses, such as ultraviolet light, nutrient deprivation, and oxygen deprivation. HSPs act as chaperones to other cellular proteins (called client proteins) and facilitate their proper folding and repair, and aid in the refolding of misfolded client proteins. There are several known families of HSPs, each having its own set of client proteins. The Hsp90 family is one of the most abundant HSP families, accounting for about 1-2% of proteins in a cell that is not under stress and increasing to about 4-6% in a cell under stress. Inhibition of Hsp90 results in degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It is now found that combination treatment of radiotherapy with certain triazolone Hsp90 inhibitors is surprisingly effective at treating subjects with cancer, e.g., colon carcinoma, colorectal cancer, gastric cancer, hepatocellular cancer, pancreatic cancer, breast cancer, ovarian cancer, prostate cancer, stomach cancer, cervical cancer, small cell lung carcinoma, non-small cell lung cancer, lung cancer and multiple myeloma. The particular therapies disclosed herein demonstrate significant anticancer effects with minimal side effects.

The present method utilizes triazolone compounds which inhibit the activity of Hsp90 and are useful in the treatment of cancer, particularly colon carcinoma, colorectal cancer, gastric cancer, hepatocellular cancer, pancreatic cancer, breast cancer, ovarian cancer, prostate cancer, stomach cancer, cervical cancer, small cell lung carcinoma, non-small cell lung cancer, and multiple myeloma, in combination with radiotherapy. A method of treating a subject with cancer includes the step of administering to the subject an Hsp90 inhibitor described herein in combination with radiotherapy. In one embodiment, the method includes treating a subject who is being or has been treated with a chemotherapeutic agent with an Hsp90 inhibitor in combination with radiotherapy. In one embodiment, the administration of the Hsp90 inhibitor and the radiotherapy are done concurrently. In another embodiment, the administration of the Hsp90 inhibitor and the radiotherapy are done separately. In another embodiment, the administration of the Hsp90 inhibitor and the radiotherapy are done sequentially. In any one of these embodiments, the Hsp90 inhibitor is a compound represented by formulae (I) or (Ia), or a compound in Table 1 or 2, or a tautomer, or a pharmaceutically acceptable salt thereof.

In one embodiment, a method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy. In one embodiment, the triazolone compound is administered synergistically with the radiotherapy for the treatment of cancer. In one embodiment, the synergistic treatment is for cervical cancer. In one embodiment, the synergistic treatment is for lung cancer. In one embodiment, the synergistic treatment is for breast cancer. In one embodiment, the synergistic treatment is for colon cancer. In one embodiment, the synergistic treatment is for non-small cell lung cancer.

In one embodiment, a method of treating a subject with cancer includes administering to the subject a synergistic amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy. In one embodiment, the treatment is for a subject with cervical cancer. In one embodiment, the treatment is for a subject with lung cancer. In one embodiment, the treatment is for a subject with breast cancer. In one embodiment, the treatment is for a subject with colon cancer. In one embodiment, the treatment is for a subject with non-small cell lung cancer.

In one embodiment, a method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy. In one embodiment, the triazolone compound is administered synergistically with the radiotherapy for the treatment of cancer. In one embodiment, the synergistic treatment is for cervical cancer. In one embodiment, the synergistic treatment is for lung cancer. In one embodiment, the synergistic treatment is for breast cancer. In one embodiment, the synergistic treatment is for colon cancer.

In one embodiment, a method of treating a subject with cancer includes administering to the subject a synergistic amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy. In one embodiment, the triazolone compound is administered synergistically with the radiotherapy for the treatment of cancer. In one embodiment, the synergistic treatment is for cervical cancer. In one embodiment, the treatment is for a subject with cervical cancer. In one embodiment, the treatment is for a subject with lung cancer. In one embodiment, the treatment is for a subject with breast cancer. In one embodiment, the treatment is for a subject with colon cancer. In one embodiment, the treatment is for a subject with non-small cell lung cancer.

In one embodiment, the invention includes the use of an Hsp90 inhibitor described herein for the manufacture of a medicament for treating cancer in combination with radiotherapy. In one embodiment, the cancer is cervical cancer. In one embodiment, the cancer is lung cancer. In one embodiment, the cancer is breast cancer. In one embodiment, the cancer is colon cancer.

In certain embodiments, the combination treatment utilizing an Hsp90 compound described herein with radiotherapy may help to prevent or reduce the development of relapsed or refractory cancer. In this embodiment, the compounds described herein may allow a reduced dose of radiotherapy given to a subject, because the Hsp90 inhibitor may potentiate or enhance the cytotoxic effect of radiotherapy, or the Hsp90 inhibitor may sensitize the cancer or tumor cells to the treatment of radiotherapy.

### BRIEF DESCRIPTION OF THE FIGURES

FIG. 1 describes nude mouse xenograft study to determine the effect of Compound 1 on the in vivo growth rate of HeLaHL human cervical carcinoma tumor cells when dosed in combination with fractionated ionizing radiation of 2 Gy.

FIG. 2 describes a nude mouse xenograft study to determine the effect of Compound 1 on the in vivo growth rate of HeLaHL human cervical carcinoma tumor cells when dosed in combination with fractionated ionizing radiation of 4 Gy.

### DETAILED DESCRIPTION OF THE INVENTION

Unless otherwise specified, the below terms used herein are defined as follows:

As used herein, the term “alkyl” means a saturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds of this invention may be optionally substituted with one or more substituents.

As used herein, the term “alkenyl” means a straight chain or branched, non-cyclic hydrocarbon having from 2 to 10 carbon atoms and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, and the like. Alkenyl groups included in compounds of the invention may be optionally substituted with one or more substituents.

As used herein, the term “alkynyl” means a straight chain or branched, non-cyclic hydrocarbon having from 2 to 10 carbon atoms and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkynyl groups included in compounds of the invention may be optionally substituted with one or more substituents.

As used herein, the term “cycloalkyl” means a saturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, and the like. Cycloalkyl groups included in compounds of the invention may be optionally substituted with one or more substituents.

As used herein, the term “cycloalkenyl” means a mono- or polycyclic, non-aromatic hydrocarbon having at least one carbon-carbon double bond in the cyclic system and having from 3 to 20 carbon atoms. Representative cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl, 1,2,3,4,5,8-hexahydronaphthalenyl, and the like. Cycloalkenyl groups included in compounds of the invention may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups included in compounds of this invention may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl and a “lower alkenyl” or “lower alkynyl” refers to an alkenyl or alkynyl radical having from 2 to 4 carbon atoms.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker. Alkoxy groups included in compounds of this invention may be optionally substituted with one or more substituents.

As used herein, a “haloalkoxy” is a haloalkyl group which is attached to another moiety via an oxygen linker.

As used herein, the term an “aromatic ring” or “aryl” means a mono- or polycyclic hydrocarbon, containing from 6 to 15 carbon atoms, in which at least one ring is aromatic. Examples of suitable aryl groups include phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds of this invention may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds of this invention may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds of this invention may be optionally substituted with one or more substituents. Only stable isomers of such substituted heterocyclic groups are contemplated in this definition.

As used herein, the term “heteroaromatic”, “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzol[1,3]dioxolyl, benzol[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds of this invention may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds of this invention may be optionally substituted with one or more substituents.

As used herein, the term “halogen” or “halo” means —F, —Cl, —Br or —I.

As used herein the term “heteroalkyl” means a straight or branched alkyl group wherein one or more of the internal carbon atoms in the chain is replaced by a heteroatom. For example, a heteroalkyl is represented by the formula —[CH₂]ₓ—Z—[CH₂]_{y}[CH₃], wherein x is a positive integer and y is zero or a positive integer, Z is O, NR, S, S(O), or S(O)₂, and wherein replacement of the carbon atom does not result in a unstable compound. Heteroalkyl groups included in compounds of this invention may be optionally substituted with one or more substituents.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound of the invention without significantly adversely affecting the reactivity or biological activity of the compound of the invention. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR³³, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹, —NR³³S(O)ₖNR²⁸R²⁹, guanidino, —C(O)SR³¹, —C(S)SR³¹, —C(NR³²)SR³¹, —OC(O)OR³¹, —OC(S)OR³¹, —OC(NR³²)OR³¹, —SC(O)R³³, —SC(O)OR³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³².

Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted.

Each R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³¹ or R³³ is optionally and independently unsubstituted.

Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₚR³³, or —S(O)ₚNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted.

The variable k is 0, 1 or 2.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, the term “compound(s) of this invention” “triazolone compound”, or similar terms refers to a compound of any one of formulae (I) or (Ia) or a compound in Table 1 or 2, or a pharmaceutically acceptable salt thereof.

As used herein, the term “pharmaceutically acceptable salt” refers to a salt prepared from a compound of any one of formulae (I) or (Ia) or a compound in Table for 2 having an acidic functional group, such as a carboxylic acid functional group, and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of any one of formulae (I) or (Ia) or a compound in Table 1 or 2 having a basic functional group, such as an amine functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

A pharmaceutically acceptable carrier may contain inert ingredients which do not unduly inhibit the biological activity of the compound(s). The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound of this invention which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used. In cases where no amount is expressly noted, an effective amount should be assumed. Non-limiting examples of an effective amount of a compound of the invention are provided herein below. In a specific embodiment, a method of treating, managing, or ameliorating cancer, or one or more symptoms thereof, including administering to a subject in need thereof a dose of at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every day, once every 2 days, once every 3 days, once every 4 days, once every 5 days, once every 6 days, once every 7 days, once every 8 days, once every 10 days, once every two weeks, once every three weeks, or once a month. The daily dose can be administered in a single portion. Alternatively, the daily dose can be divided into portions (typically equal portions) administered two times, three times, four times or more per day.

The dosage of a therapeutic agent other than a compound of the triazolone compound described herein, which has been or is currently being used to treat, manage, or ameliorate cancer, or one or more symptoms thereof, can be used in the combination therapies of the invention. Preferably, the dosage of each individual therapeutic agent used in the combination therapy is lower than the dose of an individual therapeutic agent when given independently to treat, manage, or ameliorate a disease or disorder, or one or more symptoms thereof. The recommended dosages of therapeutic agents currently used for the treatment, management, or amelioration of a disease or disorder, or one or more symptoms thereof, can obtained from any reference in the art. See, e.g., GOODMAN & GILMAN'S THE PHARMACOLOGICAL BASIS OF BASIS OF THERAPEUTICS 9^{TH} ED, (Hardman, et al., Eds., NY: Mc-Graw-Hill (1996)); PHYSICIAN'S DESK REFERENCE 57^{TH} ED. (Medical Economics Co., Inc., Montvale, N.J. (2003)).

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., cancer, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation.

As used herein, the terms “therapeutic agent” and “therapeutic agents” refer to any agent(s) that can be used in the treatment of a disease or disorder, e.g. cancer, or one or more symptoms thereof. In certain embodiments, the term “therapeutic agent” refers to a compound of the invention. In certain other embodiments, the term “therapeutic agent” does not refer to a compound of the invention. Preferably, a therapeutic agent is an agent that is known to be useful for, or has been or is currently being used for the treatment of a disease or disorder, e.g., cancer, particularly colon carcinoma, breast cancer, cervical cancer, small cell lung carcinoma, or non-small cell lung cancer, or one or more symptoms thereof.

As used herein, the term “potentiate” or “potentiating”, “sensitize” or “sensitizing”, means, in the context of this application to enhance or increase the effect of, for example, a drug, or to promote or strengthen, for example, a biochemical or physiological action or effect.

As used herein, the term “synergistic” refers to a combination of a compound of the invention and another therapeutic agent, which, when taken together, is more effective than the additive effects of the individual therapies. A synergistic effect of a combination of therapies (e.g., a combination of therapeutic agents) may permit the use of lower dosages of one or more of the therapeutic agent(s) and/or less frequent administration of the agent(s) to a subject with a disease or disorder, e.g., cancer. The combination therapy of triazolone compounds described herein with radiotherapy may permit, among other things, less frequent administration of the therapies. The ability to utilize lower dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. cancer. Finally, a synergistic effect of a combination of therapies may avoid or reduce adverse or unwanted side effects associated with the use of either therapeutic agent alone.

As used herein, the term “in combination” refers to the use of more than one therapeutic agent. The use of the term “in combination” does not restrict the order in which the therapeutic agents are administered to a subject afflicted with cancer. A first therapeutic agent, such as a compound of the invention, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent or treatment, such as an anti-cancer agent or radiotherapy, to a subject with cancer, particularly, cervical cancer, lung cancer, non-small cell lung cancer, breast cancer, or colon cancer.

As used herein, the terms “therapies” and “therapy” can refer to any protocol(s), method(s), and/or agent(s) that can be used in the prevention, treatment, management, or amelioration of cancer.

A used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

The compounds of the invention are defined herein by their chemical structures and/or chemical names. Where a compound is referred to by both a chemical structure and a chemical name, and the chemical structure and chemical name conflict, the chemical structure is determinative of the compound's identity.

Only those choices and combinations of substituents that result in a stable structure are contemplated. Such choices and combinations will be apparent to those of ordinary skill in the art and may be determined without undue experimentation.

Radiation therapy or radiotherapy is the medical use of ionizing radiation as part of cancer treatment to control malignant cells. It is used as palliative treatment or as therapeutic treatment. Radiotherapy is accepted as an important standard therapy for treating various types of cancers, and may be used for curative or adjuvant treatment, but its success as an adjuvant with another treatment, or to improve the efficacy of the other treatment, cannot reasonably be predicted to any degree of certainty.

As used herein, the term “radiotherapy” or “radiation therapy” is used for the treatment of diseases of oncological nature with ionizing radiation. Ionizing radiation deposits energy that injures or destroys cells in the area being treated (the target tissue) by damaging their genetic material, making it impossible for these cells to continue to grow. Radiotherapy may be used to treat localized solid tumors cancers of the skin, tongue, larynx, brain, breast, lung or uterine cervix. It can also be used to treat leukemia and lymphoma, i.e., cancers of the blood-forming cells and lymphatic system, respectively. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiotherapy. Gamma rays are another form of photons used in radiotherapy. Gamma rays are produced spontaneously as certain elements (such as radium, uranium, and cobalt 60) release radiation as they decompose, or decay.

Another technique for delivering radiation to cancer cells is to place radioactive implants directly in a tumor or body cavity. This is called internal radiotherapy. Brachytherapy, interstitial irradiation, and intracavitary irradiation are types of internal radiotherapy. In this treatment, the radiation dose is concentrated in a small area, and the patient stays in the hospital for a few days. Internal radiotherapy is frequently used for cancers of the tongue, uterus, and cervix.

A further technique is intra-operative irradiation, in which a large dose of external radiation is directed at the tumor and surrounding tissue during surgery. Another approach is particle beam radiation therapy. This type of therapy differs from photon radiotherapy in that it involves the use of fast-moving subatomic particles to treat localized cancers. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation. Radio-sensitizers make the tumor cells more likely to be damaged, and radio-protectors protect normal tissues from the effects of radiation.

A person of ordinary skill in the radiotherapy art knows how to determine an appropriate dosing and application schedule, depending on the nature of the disease and the constitution of the patient. In particular, the person knows how to assess dose-limiting toxicity (DLT) and how to determine the maximum tolerated dose (MTD) accordingly.

More particularly, the amount of radiation used in photon radiation therapy is measured in gray (Gy), and varies depending on the type and stage of cancer being treated. For curative cases, the typical dose for a solid epithelial tumor ranges from 60 to 80 Gy, while lymphomas are treated with 20 to 40 Gy.

Preventative (adjuvant) doses are typically around 45-60 Gy in 1.8-2 Gy fractions (for breast, head, and neck cancers). Many other factors are considered by radiation oncologists when selecting a dose, including whether the patient is receiving chemotherapy, patient co-morbidities, whether radiation therapy is being administered before or after surgery, and the degree of success of surgery.

Moreover, the total dose is often fractionated (spread out over time) for several important reasons. Fractionation allows normal cells time to recover, while tumor cells are generally less efficient in repair between fractions. Fractionation also allows tumor cells that were in a relatively radioresistant phase of the cell cycle during one treatment to cycle into a sensitive phase of the cycle before the next fraction is given. Similarly, tumor cells that were chronically or acutely hypoxic (and therefore more radioresistant) may reoxygenate between fractions, improving the tumor cell kill. Fractionation regimes are individualized between different radiotherapy centers and even between individual doctors. In North America, Australia, and Europe, the typical fractionation schedule for adults is 1.8 to 2 Gy per day, five days a week. In some cancer types, prolongation of the fraction schedule over too long can allow for the tumor to begin repopulating, and for these tumor types, including head-and-neck and cervical squamous cell cancers, radiation treatment is preferably completed within a certain amount of time. For children, a typical fraction size may be 1.5 to 1.8 Gy per day, as smaller fraction sizes are associated with reduced incidence and severity of late-onset side effects in normal tissues.

In some cases, two fractions per day are used near the end of a course of treatment. This schedule, known as a concomitant boost regimen or hyperfractionation, is used on tumors that regenerate more quickly when they are smaller. In particular, tumors in the head-and-neck demonstrate this behavior.

One of the best-known alternative fractionation schedules is Continuous Hyperfractionated Accelerated Radiotherapy (CHART). CHART, used to treat lung cancer, consists of three smaller fractions per day.

Another increasingly well-known alternative fractionation schedule, used to treat breast cancer, is called Accelerated Partial Breast Irradiation (APBI). APBI can be performed with either brachytherapy or with external beam radiation. APBI normally involves two high-dose fractions per day for five days, compared to whole breast irradiation, in which a single, smaller fraction is given five times a week over a six-to-seven-week period.

Implants can be fractionated over minutes or hours, or they can be permanent seeds which slowly deliver radiation until they become inactive.

The invention can be understood more fully by reference to the following detailed description and illustrative examples, which are intended to exemplify non-limiting embodiments of the invention.

The present invention utilizes triazolone compounds represented by Formulae (I) or (Ia):

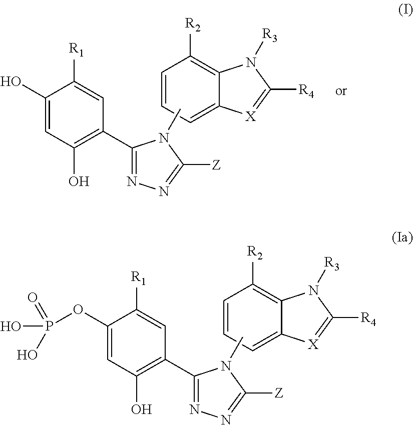

or a tautomer, or a pharmaceutically acceptable salt thereof, wherein:
Z is OH, SH, or NH₂;
X is CR₄ or N;

R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R_{D}, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄.

In another embodiment, in formula (I) or (Ia), X is N.

In another embodiment, in formula (I) or (Ia), R₁ is selected from the group consisting of —H, lower alkyl, lower alkoxy, lower cycloalkyl, and lower cycloalkoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy.

In another embodiment, in formula (I) or (Ia), R₃ is selected from the group consisting of —H, a lower alkyl, a lower cycloalkyl, —C(O)N(R₂₇)₂, and —C(O)OH, wherein R₂₇ is —H or a lower alkyl.

In another embodiment, in formula (I) or (Ia), R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂.

In one embodiment, R₄ is H or a lower alkyl.

In another embodiment, in formula (I) or (Ia), R₄ is selected from the group consisting of —H, methyl, ethyl, propyl, isopropyl or cyclopropyl.

In another embodiment, in formula (I) or (Ia), R₁ is selected from the group consisting of —H, —OH, —SH, —NH₂, a lower alkoxy and a lower alkyl amino.

In another embodiment, in formula (I) or (Ia), R₁ is selected from the group consisting of —H, —OH, methoxy and ethoxy.

In another embodiment, in formula (I) or (Ia), Z is —OH.

In another embodiment, in formula (I) or (Ia), Z is —SH.

In another embodiment, in formula (I) or (Ia), R₂ is selected from the group consisting of —H, —OH, —SH, —NH₂, a lower alkoxy and a lower alkyl amino.

In another embodiment, in formula (I) or (Ia), R₂ is selected from the group consisting of —H, —OH, methoxy, and ethoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is OH.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is SH.

In another embodiment, the compound is selected from the group consisting of:
3-(2,4-dihydroxy-5-ethyl-phenyl)-4-(1,3-dimethyl-indol-5-yl)-5-hydroxy-[1,2,4]triazole,
3-(2,4-dihydroxy-5-isopropyl-phenyl)-4-(1,3-dimethyl-indol-5-yl)-5-hydroxy-[1,2,4]triazole,
3-(2,4-dihydroxy-5-isopropyl-phenyl)-4-(1-methyl-indol-5-yl)-5-hydroxy-[1,2,4]triazole,
3-(2,4-dihydroxy-5-isopropyl-phenyl)-4-(1-isopropyl-indol-4-yl)-5-hydroxy-[1,2,4]triazole,
3-(2,4-dihydroxy-5-isopropyl-phenyl)-4-(1-methyl-indazol-5-yl)-5-mercapto-[1,2,4]triazole,
3-(2,4-dihydroxy-5-isopropyl-phenyl)-4-(1-methyl-indazol-6-yl)-5-mercapto-[1,2,4]triazole,
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole,
3-(2,4-dihydroxy-5-isopropyl-phenyl)-4-(1-propyl-indol-5-yl)-5-mercapto-[1,2,4]triazole, or a tautomer, or a pharmaceutically acceptable salt thereof.

In another embodiment, in formula (I) or (Ia), X is N.

In another embodiment, the compound is selected from the group consisting of
3-(2,4-dihydroxy-5-ethyl-phenyl)-4-(1-ethyl-benzimidazol-4-yl)-5-mercapto-[1,2,4]triazole,
3-(2,4-dihydroxy-5-ethyl-phenyl)-4-(1-ethyl-benzimidazol-4-yl)-5-mercapto-[1,2,4]triazole HCL salt,
3-(2,4-dihydroxy-5-ethyl-phenyl)-4-(2-methyl-3-ethyl-benzimidazol-5-yl)-5-mercapto-[1,2,4]triazole,
3-(2,4-dihydroxy-5-ethyl-phenyl)-4-(1-ethyl-2-methyl-benzimidazol-5-yl)-5-mercapto-[1,2,4]triazole,
3-(2,4-dihydroxy-5-isopropyl-phenyl)-4-(1-methyl-2-trifluoromethyl-benzimidazol-5-yl)-5-mercapto-[1,2,4]triazole, or a tautomer, or a pharmaceutically acceptable salt thereof.

### i) Exemplary Compounds of the Invention

Exemplary compounds described herein are depicted in Table 1 below, including tautomers or pharmaceutically acceptable salts.

TABLE 1

| | Structure | Tautomeric Structure | Name |
|---|---|---|---|
| 1 | 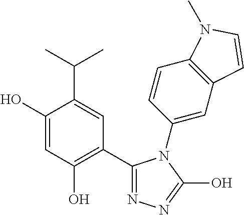 [SMILES: CC(C)c1cc(-c2nnc(O)n2-c2ccc3c(ccn3C)c2)c(O)cc1O] | 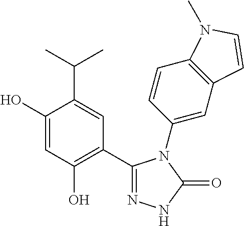 [SMILES: CC(C)c1cc(-c2n[nH]c(=O)n2-c2ccc3c(ccn3C)c2)c(O)cc1O] | 3-(2,4-dihydroxy-5- isopropyl-phenyl)-4- (1-methyl-indol-5-yl)- 5-hydroxy-[1,2,4] triazole |
| 2 | 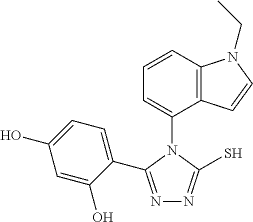 [SMILES: CCn1ccc2c(-n3c(S)nnc3-c3ccc(O)cc3O)cccc21] | 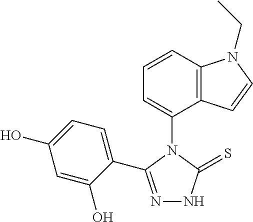 [SMILES: CCn1ccc2c(-n3c(-c4ccc(O)cc4O)n[nH]c3=S)cccc21] | 3-(2,4- Dihydroxyphenyl)-4- (1-ethyl-indol-4-yl)-5- mercapto-[1,2,4] triazole |
| 3 | 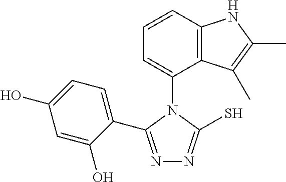 [SMILES: Cc1[nH]c2cccc(-n3c(S)nnc3-c3ccc(O)cc3O)c2c1C] | 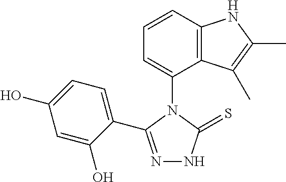 [SMILES: Cc1[nH]c2cccc(-n3c(-c4ccc(O)cc4O)n[nH]c3=S)c2c1C] | 3-(2,4-Dihydroxy- phenyl)-4-(2,3- dimethyl-1H-indol-4- yl)-5-mercapto-[1,2,4] triazole |
| 4 | 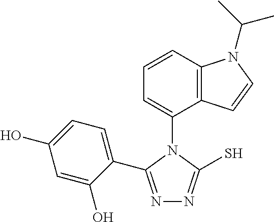 [SMILES: CC(C)n1ccc2c(-n3c(S)nnc3-c3ccc(O)cc3O)cccc21] | 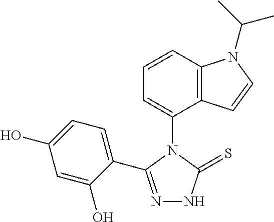 [SMILES: CC(C)n1ccc2c(-n3c(-c4ccc(O)cc4O)n[nH]c3=S)cccc21] | 3-(2,4- Dihydroxyphenyl)-4- (1-isopropyl-indol-4- yl)-5-mercapto-[1,2,4] triazole |
| 5 | 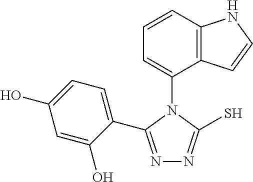 [SMILES: Oc1ccc(-c2nnc(S)n2-c2cccc3[nH]ccc23)c(O)c1] | 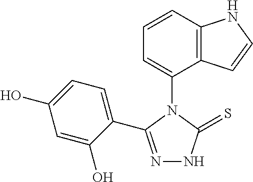 [SMILES: Oc1ccc(-c2n[nH]c(=S)n2-c2cccc3[nH]ccc23)c(O)c1] | 3-(2,4-Dihydroxy- phenyl)-4-(indol-4- yl)-5-mercapto-[1,2,4] triazole |
| 6 | 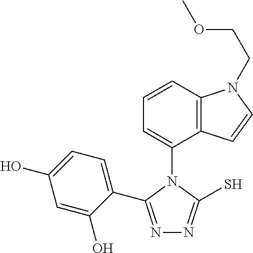 [SMILES: COCCn1ccc2c(-n3c(S)nnc3-c3ccc(O)cc3O)cccc21] | 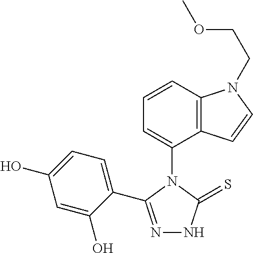 [SMILES: COCCn1ccc2c(-n3c(-c4ccc(O)cc4O)n[nH]c3=S)cccc21] | 3-(2,4-Dihydroxy- phenyl)-4-[1-(2- methoxyethoxy)- indol-4-yl]-5- mercapto-[1,2,4] triazole |
| 7 | 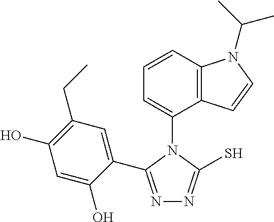 [SMILES: CCc1cc(-c2nnc(S)n2-c2cccc3c2ccn3C(C)C)c(O)cc1O] | 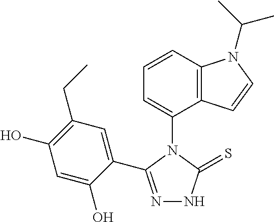 [SMILES: CCc1cc(-c2n[nH]c(=S)n2-c2cccc3c2ccn3C(C)C)c(O)cc1O] | 3-(2,4-Dihydroxy-5- ethyl-phenyl)-4-(1- isopropyl-indol-4-yl)- 5-mercapto-[1,2,4] triazole |
| 8 | 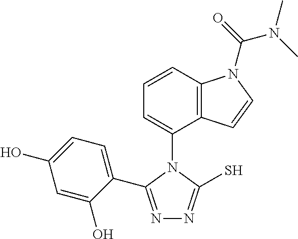 [SMILES: CN(C)C(=O)n1ccc2c(-n3c(S)nnc3-c3ccc(O)cc3O)cccc21] | 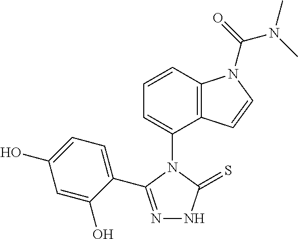 [SMILES: CN(C)C(=O)n1ccc2c(-n3c(-c4ccc(O)cc4O)n[nH]c3=S)cccc21] | 3-(2,4-Dihydroxy-5- ethyl-phenyl)-4-[1- (dimethyl- carbamoyl)-indol-4- yl]-5-mercapto-[1,2,4] triazole |
| 9 | 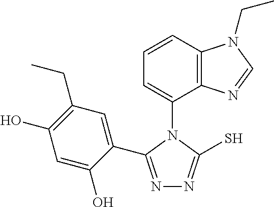 [SMILES: CCc1cc(-c2nnc(S)n2-c2cccc3c2ncn3CC)c(O)cc1O] | 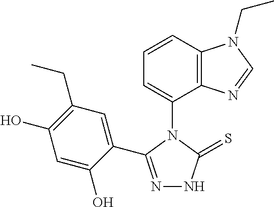 [SMILES: CCc1cc(-c2n[nH]c(=S)n2-c2cccc3c2ncn3CC)c(O)cc1O] | 3-(2,4-Dihydroxy-5- ethyl-phenyl)-4-(1- ethyl-benzoimidazol- 4-yl)-5-mercapto- [1,2,4] triazole |
| 10 | 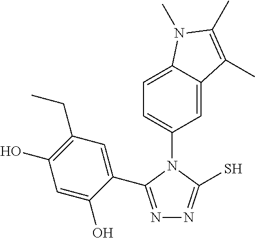 [SMILES: CCc1cc(-c2nnc(S)n2-c2ccc3c(c2)c(C)c(C)n3C)c(O)cc1O] | 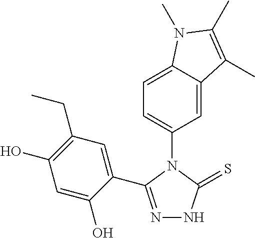 [SMILES: CCc1cc(-c2n[nH]c(=S)n2-c2ccc3c(c2)c(C)c(C)n3C)c(O)cc1O] | 3-(2,4-Dihydroxy-5- ethyl-phenyl)-4- (1,2,3-trimethyl-indol- 5-yl)-5-mercapto- [1,2,4] triazole |
| 11 | 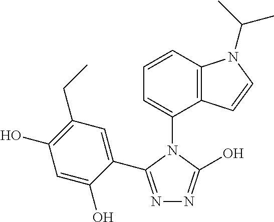 [SMILES: CCc1cc(-c2nnc(O)n2-c2cccc3c2ccn3C(C)C)c(O)cc1O] | 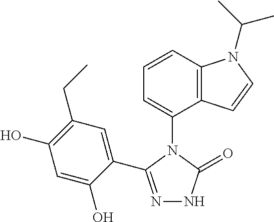 [SMILES: CCc1cc(-c2n[nH]c(=O)n2-c2cccc3c2ccn3C(C)C)c(O)cc1O] | 3-(2,4-Dihydroxy-5- ethyl-phenyl)-4-(1- isopropyl-indol-3-yl)- 5-hydroxy-[1,2,4] triazole |
| 12 | 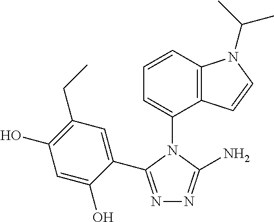 [SMILES: CCc1cc(-c2nnc(N)n2-c2cccc3c2ccn3C(C)C)c(O)cc1O] | 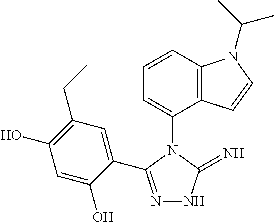 [SMILES: CCc1cc(-c2n[nH]c(=N)n2-c2cccc3c2ccn3C(C)C)c(O)cc1O] | 3-(2,4-Dihydroxy-5- ethyl-phenyl)-4-(1- isopropyl-indol-4-yl)- 5-amino-[1,2,4] triazole |
| 15 | 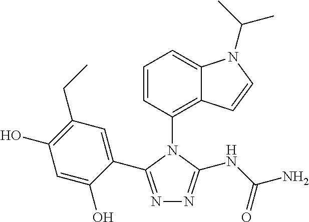 [SMILES: CCc1cc(-c2nnc(NC(N)=O)n2-c2cccc3c2ccn3C(C)C)c(O)cc1O] | | 3-(2,4-Dihydroxy-5- ethyl-phenyl)-4-(1- isopropyl-indol-4-yl)- 5-ureido-[1,2,4] triazole |
| 16 | 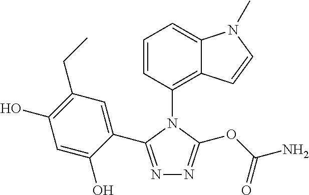 [SMILES: CCc1cc(-c2nnc(OC(N)=O)n2-c2cccc3c2ccn3C)c(O)cc1O] | | 3-(2,4-Dihydroxy-5- ethyl-phenyl)-4-(1- methyl-indol-4-yl)-5- carbamoyloxy-[1,2,4] triazole |
| 17 | 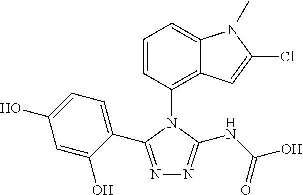 [SMILES: Cn1c(Cl)cc2c(-n3c(NC(=O)O)nnc3-c3ccc(O)cc3O)cccc21] | | 3-(2,4-Dihydroxy- phenyl)-4-(1-methyl- 2-chloro-indol-4-yl)- 5-carbamoyloxy- [1,2,4] triazole |
| 18 | 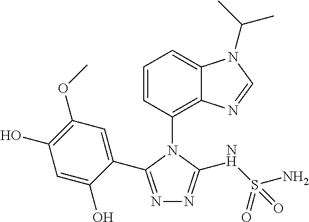 [SMILES: COc1cc(-c2nnc(NS(N)(=O)=O)n2-c2cccc3c2ncn3C(C)C)c(O)cc1O] | | 3-(2,4-Dihydroxy-5- methoxy-phenyl)-4- (1-isopropyl- benzoimidazol-4-yl)- 5-(sulfamoylamino)- [1,2,4] triazole |
| 20 | 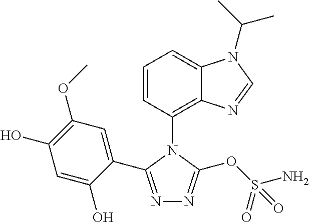 [SMILES: COc1cc(-c2nnc(OS(N)(=O)=O)n2-c2cccc3c2ncn3C(C)C)c(O)cc1O] | | 3-(2,4-Dihydroxy-5- methoxy-phenyl)-4- (1-isopropyl- benzoimidazol-4-yl)- 5-(sulfamoyloxy)- [1,2,4] triazole |
| 21 | 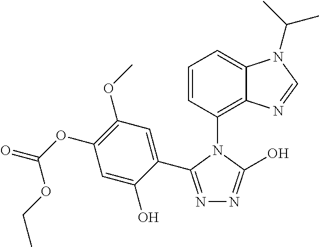 [SMILES: CCOC(=O)Oc1cc(O)c(-c2nnc(O)n2-c2cccc3c2ncn3C(C)C)cc1OC] | 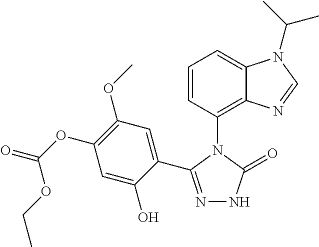 [SMILES: CCOC(=O)Oc1cc(O)c(-c2n[nH]c(=O)n2-c2cccc3c2ncn3C(C)C)cc1OC] | 3-(2-Hydroxy-4- ethoxycarbonyoxy-5- methoxy-phenyl)-4- (1-isopropyl- benzoimidazol-4-yl)- 5-hydroxy-[1,2,4] triazole |
| 22 | 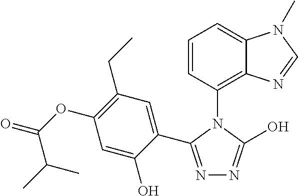 [SMILES: CCc1cc(-c2nnc(O)n2-c2cccc3c2ncn3C)c(O)cc1OC(=O)C(C)C] | 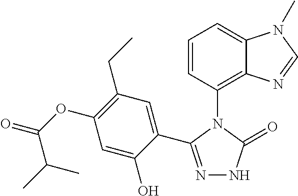 [SMILES: CCc1cc(-c2n[nH]c(=O)n2-c2cccc3c2ncn3C)c(O)cc1OC(=O)C(C)C] | 3-[2-Hydroxy-4- isobutyryloxy-5-ethyl- phenyl]-4-(1-methyl- benzo-imidazol-4-yl)- 5-hydroxy-[1,2,4] triazole |
| 23 | 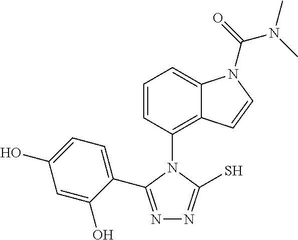 [SMILES: CN(C)C(=O)n1ccc2c(-n3c(S)nnc3-c3ccc(O)cc3O)cccc21] | 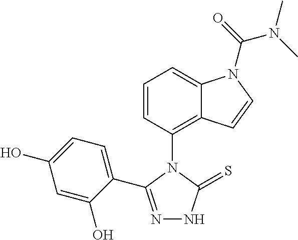 [SMILES: CN(C)C(=O)n1ccc2c(-n3c(-c4ccc(O)cc4O)n[nH]c3=S)cccc21] | 3-(2,4-Dihydroxy- phenyl)-4-(1- dimethylcarbamoyl- indol-4-yl)-5- mercapto-[1,2,4] triazole |
| 24 | 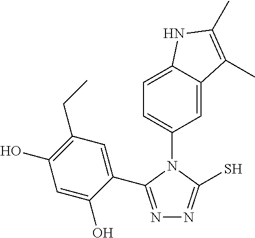 [SMILES: CCc1cc(-c2nnc(S)n2-c2ccc3[nH]c(C)c(C)c3c2)c(O)cc1O] | 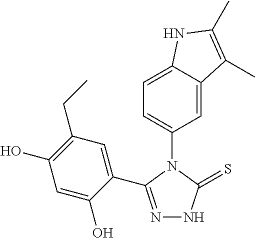 [SMILES: CCc1cc(-c2n[nH]c(=S)n2-c2ccc3[nH]c(C)c(C)c3c2)c(O)cc1O] | 3-(2,4-Dihydroxy-5- ethyl-phenyl)-4-(2,3- dimethyl-indol-5-yl)- 5-mercapto-[1,2,4] triazole |
| 25 | 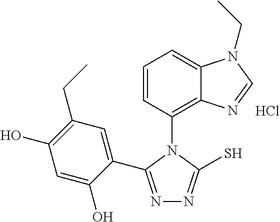 [SMILES: CCc1cc(-c2nnc(S)n2-c2cccc3c2ncn3CC)c(O)cc1O ; Cl] | 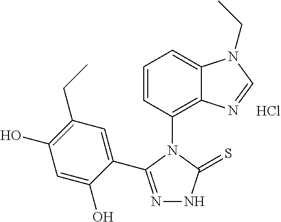 [SMILES: CCc1cc(-c2n[nH]c(=S)n2-c2cccc3c2ncn3CC)c(O)cc1O ; Cl] | 3-(2,4-Dihydroxy-5- ethyl-phenyl)-4-(1- ethyl-1H- benzoimidazol-4-yl)- 5-mercapto-[1,2,4] triazole, HCl salt |
| 26 | 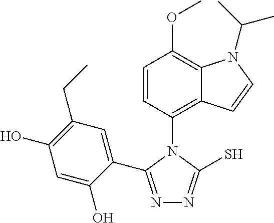 [SMILES: CCc1cc(-c2nnc(S)n2-c2ccc(OC)c3c2ccn3C(C)C)c(O)cc1O] | 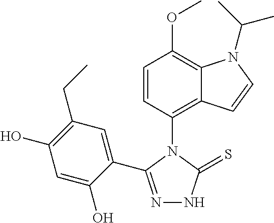 [SMILES: CCc1cc(-c2n[nH]c(=S)n2-c2ccc(OC)c3c2ccn3C(C)C)c(O)cc1O] | 3-(2,4-Dihydroxy-5- ethyl-phenyl)-4-(1- isopropyl-7-methoxy- indol-4-yl)-5- mercapto-[1,2,4] triazole |
| 27 | 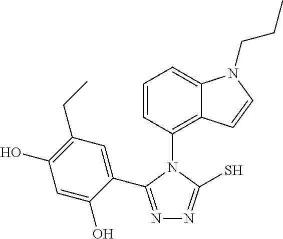 [SMILES: CCCn1ccc2c(-n3c(S)nnc3-c3cc(CC)c(O)cc3O)cccc21] | 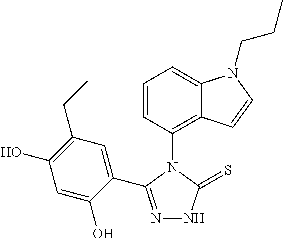 [SMILES: CCCn1ccc2c(-n3c(-c4cc(CC)c(O)cc4O)n[nH]c3=S)cccc21] | 3-(2,4-dihydroxy-5- ethyl-phenyl)-4-(1- propyl-indol-4-yl)-5- mercapto-[1,2,4] triazole |
| 28 | 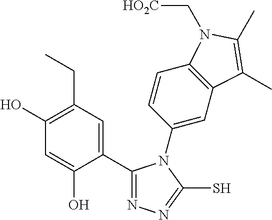 [SMILES: CCc1cc(-c2nnc(S)n2-c2ccc3c(c2)c(C)c(C)n3CC(=O)O)c(O)cc1O] | 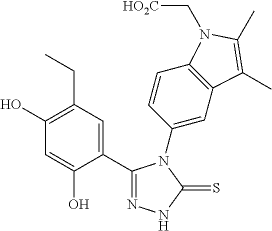 [SMILES: CCc1cc(-c2n[nH]c(=S)n2-c2ccc3c(c2)c(C)c(C)n3CC(=O)O)c(O)cc1O] | 3-(2,4-dihydroxy-5- ethyl-phenyl)-4-(1- acetyl-2,3-dimethyl- indol-5-yl)-5- mercapto-[1,2,4] triazole |
| 29 | 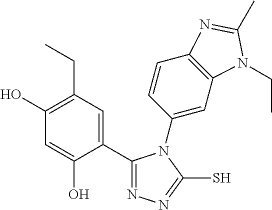 [SMILES: CCc1cc(-c2nnc(S)n2-c2ccc3nc(C)n(CC)c3c2)c(O)cc1O] | 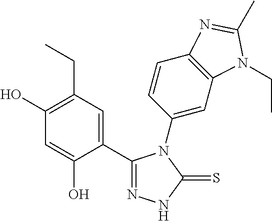 [SMILES: CCc1cc(-c2n[nH]c(=S)n2-c2ccc3nc(C)n(CC)c3c2)c(O)cc1O] | 3-(2,4-dihydroxy-5- ethyl-phenyl)-4-(2- methyl-3-ethyl- benzimidazol-5-yl)-5- mercapto-[1,2,4] triazole |
| 30 | 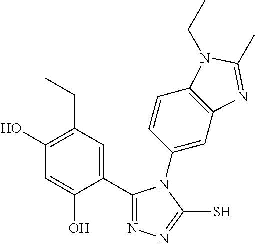 [SMILES: CCc1cc(-c2nnc(S)n2-c2ccc3c(c2)nc(C)n3CC)c(O)cc1O] | 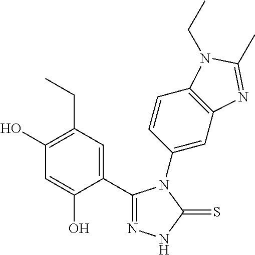 [SMILES: CCc1cc(-c2n[nH]c(=S)n2-c2ccc3c(c2)nc(C)n3CC)c(O)cc1O] | 3-(2,4-dihydroxy-5- ethyl-phenyl)-4-(1- ethyl-2-methyl- benzimidazol-5-yl)-5- mercapto-[1,2,4] triazole |
| 31 | 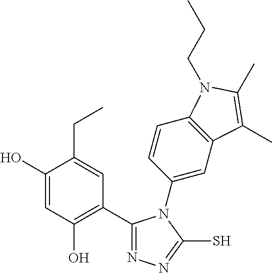 [SMILES: CCCn1c(C)c(C)c2cc(-n3c(S)nnc3-c3cc(CC)c(O)cc3O)ccc21] | 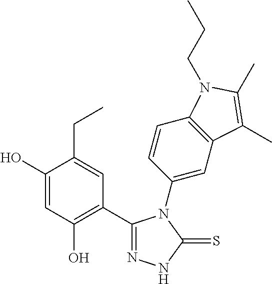 [SMILES: CCCn1c(C)c(C)c2cc(-n3c(-c4cc(CC)c(O)cc4O)n[nH]c3=S)ccc21] | 3-(2,4-dihydroxy-5- ethyl-phenyl)-4-(1- propyl-2,3-dimethyl- indol-5-yl)-5- mercapto-[1,2,4] triazole |
| 34 | 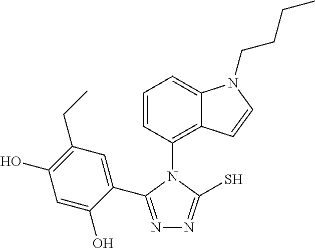 [SMILES: CCCCn1ccc2c(-n3c(S)nnc3-c3cc(CC)c(O)cc3O)cccc21] | 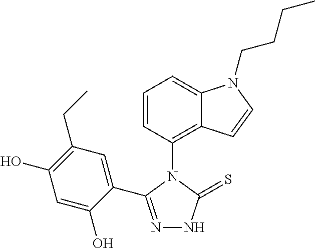 [SMILES: CCCCn1ccc2c(-n3c(-c4cc(CC)c(O)cc4O)n[nH]c3=S)cccc21] | 3-(2,4-dihydroxy-5- ethyl-phenyl)-4-(1-n- butyl-indol-4-yl)-5- mercapto-[1,2,4] triazole |
| 35 | 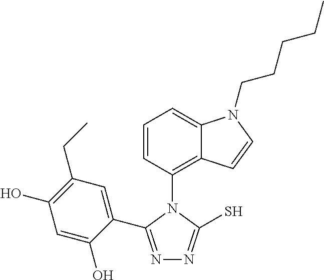 [SMILES: CCCCCn1ccc2c(-n3c(S)nnc3-c3cc(CC)c(O)cc3O)cccc21] | 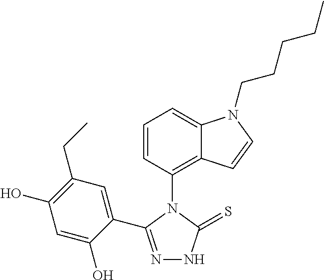 [SMILES: CCCCCn1ccc2c(-n3c(-c4cc(CC)c(O)cc4O)n[nH]c3=S)cccc21] | 3-(2,4-dihydroxy-5- ethyl-phenyl)-4-(1-n- pentyl-indol-4-yl)-5- mercapto-[1,2,4] triazole |
| 36 | 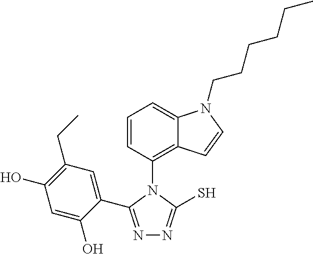 [SMILES: CCCCCCn1ccc2c(-n3c(S)nnc3-c3cc(CC)c(O)cc3O)cccc21] | 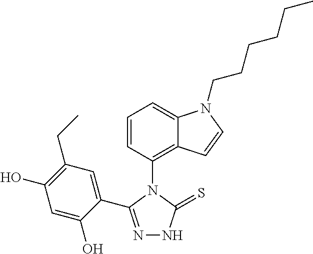 [SMILES: CCCCCCn1ccc2c(-n3c(-c4cc(CC)c(O)cc4O)n[nH]c3=S)cccc21] | 3-(2,4-dihydroxy-5- ethyl-phenyl)-4-(1-n- hexyl-indol-4-yl)-5- mercapto-[1,2,4] triazole |
| 37 | 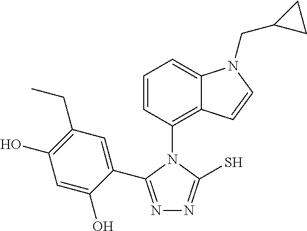 [SMILES: CCc1cc(-c2nnc(S)n2-c2cccc3c2ccn3CC2CC2)c(O)cc1O] | 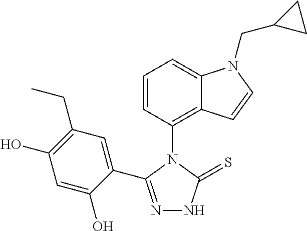 [SMILES: CCc1cc(-c2n[nH]c(=S)n2-c2cccc3c2ccn3CC2CC2)c(O)cc1O] | 3-(2,4-dihydroxy-5- cyclopropyl-phenyl)- 4-(1-(1- methylcyclopropyl)- indol-4-yl)-5- mercapto-[1,2,4] triazole |
| 38 | 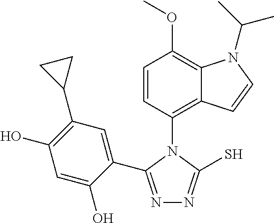 [SMILES: COc1ccc(-n2c(S)nnc2-c2cc(C3CC3)c(O)cc2O)c2ccn(C(C)C)c12] | 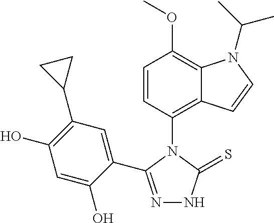 [SMILES: COc1ccc(-n2c(-c3cc(C4CC4)c(O)cc3O)n[nH]c2=S)c2ccn(C(C)C)c12] | 3-(2,4-dihydroxy-5- cyclopropyl-phenyl)- 4-(1-isopropyl-7- methoxy-indol-4-yl)- 5-mercapto-[1,2,4] triazole |
| 39 | 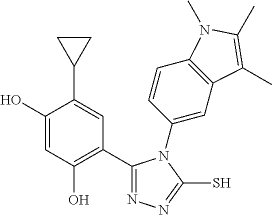 [SMILES: Cc1c(C)n(C)c2ccc(-n3c(S)nnc3-c3cc(C4CC4)c(O)cc3O)cc12] | 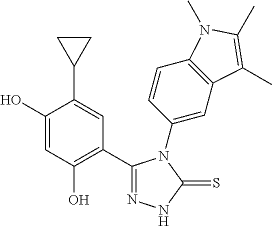 [SMILES: Cc1c(C)n(C)c2ccc(-n3c(-c4cc(C5CC5)c(O)cc4O)n[nH]c3=S)cc12] | 3-(2,4-dihydroxy-5- cyclopropyl-phenyl)- 4-(1,2,3-trimethyl- indol-5-yl)-5- mercapto-[1,2,4] triazole |
| 40 | 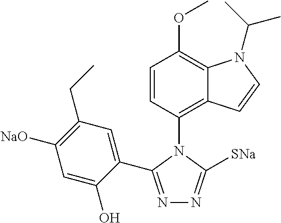 | | 3-(2,4-dihydroxy-5- ethyl-phenyl)-4-(1- isopropyl-7-methoxy- indol-4-yl)-5- mercapto-[1,2,4] triazole disodium salt |
| 41 | 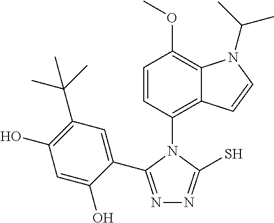 [SMILES: COc1ccc(-n2c(S)nnc2-c2cc(C(C)(C)C)c(O)cc2O)c2ccn(C(C)C)c12] | 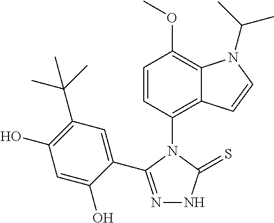 [SMILES: COc1ccc(-n2c(-c3cc(C(C)(C)C)c(O)cc3O)n[nH]c2=S)c2ccn(C(C)C)c12] | 3-(2,4-dihydroxy-5- tert-butyl-phenyl)-4- (1-isopropyl-7- methoxy-indol-4-yl)- 5-mercapto-[1,2,4] triazole |
| 42 | 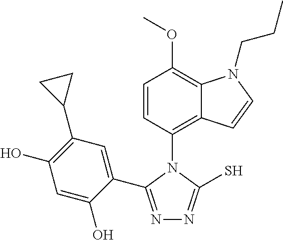 [SMILES: CCCn1ccc2c(-n3c(S)nnc3-c3cc(C4CC4)c(O)cc3O)ccc(OC)c21] | 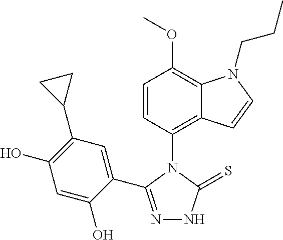 [SMILES: CCCn1ccc2c(-n3c(-c4cc(C5CC5)c(O)cc4O)n[nH]c3=S)ccc(OC)c21] | 3-(2,4-dihydroxy-5- cyclopropyl-phenyl)- 4-(1-propyl-7- methoxy-indol-4-yl)- 5-mercapto-[1,2,4] triazole |
| 43 | 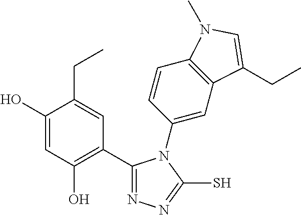 [SMILES: CCc1cc(-c2nnc(S)n2-c2ccc3c(c2)c(CC)cn3C)c(O)cc1O] | 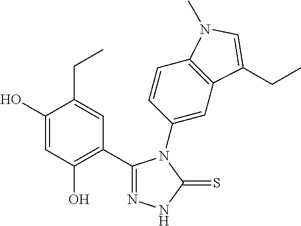 [SMILES: CCc1cc(-c2n[nH]c(=S)n2-c2ccc3c(c2)c(CC)cn3C)c(O)cc1O] | 3-(2,4-dihydroxy-5- ethyl-phenyl)-4-(1- methyl-3-ethyl-indol- 5-yl)-5-mercapto- [1,2,4] triazole |
| 44 | 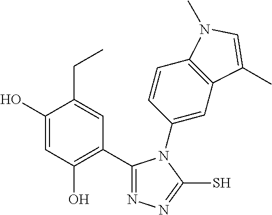 [SMILES: CCc1cc(-c2nnc(S)n2-c2ccc3c(c2)c(C)cn3C)c(O)cc1O] | 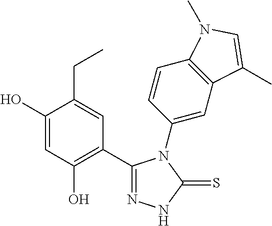 [SMILES: CCc1cc(-c2n[nH]c(=S)n2-c2ccc3c(c2)c(C)cn3C)c(O)cc1O] | 3-(2,4-dihydroxy-5- ethyl-phenyl)-4-(1,3- dimethyl-indol-5-yl)- 5-mercapto-[1,2,4] triazole |
| 45 | 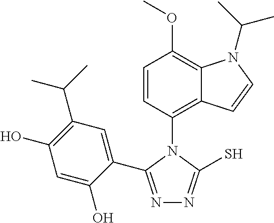 [SMILES: COc1ccc(-n2c(S)nnc2-c2cc(C(C)C)c(O)cc2O)c2ccn(C(C)C)c12] | 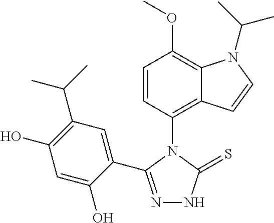 [SMILES: COc1ccc(-n2c(-c3cc(C(C)C)c(O)cc3O)n[nH]c2=S)c2ccn(C(C)C)c12] | 3-(2,4-dihydroxy-5- isopropyl-phenyl)-4- (1-isopropyl-7- methoxy-indol-4-yl)- 5-mercapto-[1,2,4] triazole |
| 46 | 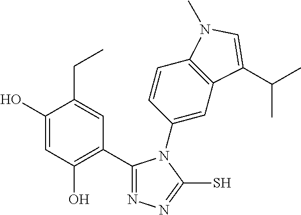 [SMILES: CCc1cc(-c2nnc(S)n2-c2ccc3c(c2)c(C(C)C)cn3C)c(O)cc1O] | 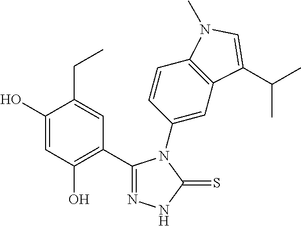 [SMILES: CCc1cc(-c2n[nH]c(=S)n2-c2ccc3c(c2)c(C(C)C)cn3C)c(O)cc1O] | 3-(2,4-dihydroxy-5- ethyl-phenyl)-4-(1- methyl-3-isopropyl- indol-5-yl)-5- mercapto-[1,2,4] triazole |
| 48 | 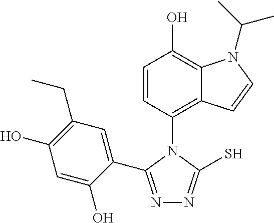 [SMILES: CCc1cc(-c2nnc(S)n2-c2ccc(O)c3c2ccn3C(C)C)c(O)cc1O] | 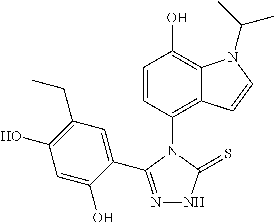 [SMILES: CCc1cc(-c2n[nH]c(=S)n2-c2ccc(O)c3c2ccn3C(C)C)c(O)cc1O] | 3-(2,4-dihydroxy-5- ethyl-phenyl)-4-(1- isopropyl-7-hydroxy- indol-4-yl)-5- mercapto-[1,2,4] triazole |
| 49 | 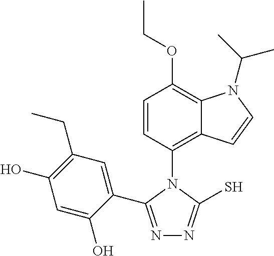 [SMILES: CCOc1ccc(-n2c(S)nnc2-c2cc(CC)c(O)cc2O)c2ccn(C(C)C)c12] | 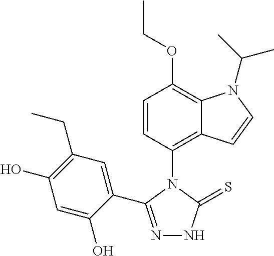 [SMILES: CCOc1ccc(-n2c(-c3cc(CC)c(O)cc3O)n[nH]c2=S)c2ccn(C(C)C)c12] | 3-(2,4-dihydroxy-5- ethyl-phenyl)-4-(1- isopropyl-7-ethoxy- indol-4-yl)-5 mercapto-[1,2,4] triazole |
| 50 | 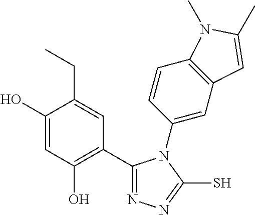 [SMILES: CCc1cc(-c2nnc(S)n2-c2ccc3c(c2)cc(C)n3C)c(O)cc1O] | 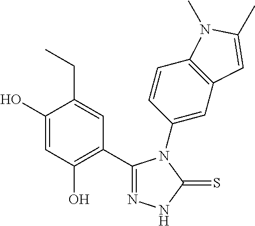 [SMILES: CCc1cc(-c2n[nH]c(=S)n2-c2ccc3c(c2)cc(C)n3C)c(O)cc1O] | 3-(2,4-dihydroxy-5- ethyl-phenyl)-4-(1,2- dimethyl-indol-5-yl)- 5-mercapto-[1,2,4] triazole |
| 51 | 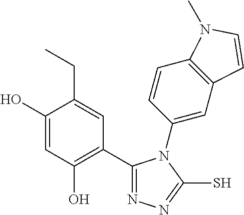 [SMILES: CCc1cc(-c2nnc(S)n2-c2ccc3c(ccn3C)c2)c(O)cc1O] | 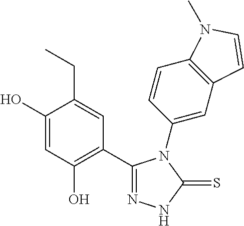 [SMILES: CCc1cc(-c2n[nH]c(=S)n2-c2ccc3c(ccn3C)c2)c(O)cc1O] | 3-(2,4-dihydroxy-5- ethyl-phenyl)-4-(N- methyl-indol-5-yl)-5- mercapto-[1,2,4] triazole |
| 55 | 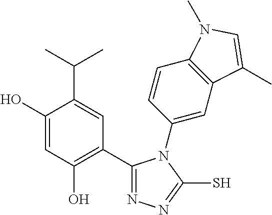 [SMILES: Cc1cn(C)c2ccc(-n3c(S)nnc3-c3cc(C(C)C)c(O)cc3O)cc12] | 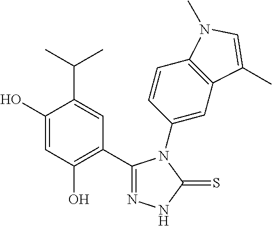 [SMILES: Cc1cn(C)c2ccc(-n3c(-c4cc(C(C)C)c(O)cc4O)n[nH]c3=S)cc12] | 3-(2,4-dihydroxy-5- isopropyl-phenyl)-4- (1,3-dimethyl-indol-5- yl)-5-mercapto-[1,2,4] triazole |
| 56 | 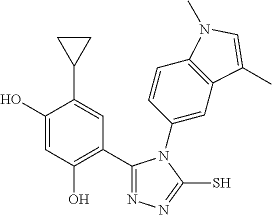 [SMILES: Cc1cn(C)c2ccc(-n3c(S)nnc3-c3cc(C4CC4)c(O)cc3O)cc12] | 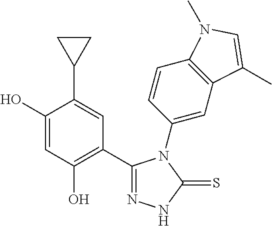 [SMILES: Cc1cn(C)c2ccc(-n3c(-c4cc(C5CC5)c(O)cc4O)n[nH]c3=S)cc12] | 3-(2,4-dihydroxy-5- cyclopropyl-phenyl)- 4-(1,3-dimethyl-indol- 5-yl)-5-mercapto- [1,2,4] triazole |
| 57 | 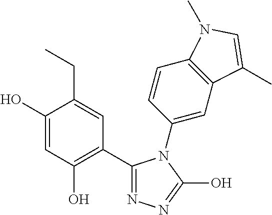 [SMILES: CCc1cc(-c2nnc(O)n2-c2ccc3c(c2)c(C)cn3C)c(O)cc1O] | 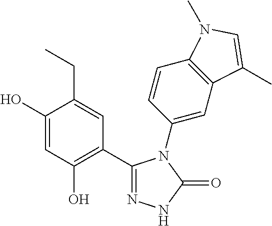 [SMILES: CCc1cc(-c2n[nH]c(=O)n2-c2ccc3c(c2)c(C)cn3C)c(O)cc1O] | 3-(2,4-dihydroxy-5- ethyl-phenyl)-4-(1,3- dimethyl-indol-5-yl)- 5-hydroxy-[1,2,4] triazole |
| 58 | 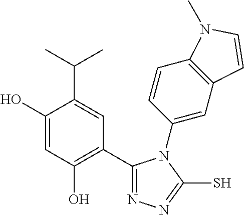 [SMILES: CC(C)c1cc(-c2nnc(S)n2-c2ccc3c(ccn3C)c2)c(O)cc1O] | 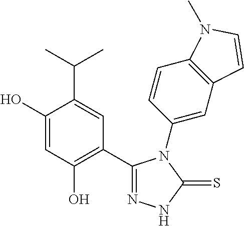 [SMILES: CC(C)c1cc(-c2n[nH]c(=S)n2-c2ccc3c(ccn3C)c2)c(O)cc1O] | 3-(2,4-dihydroxy-5- isopropyl-phenyl)-4- (N-methyl-indol-5- yl)-5-mercapto-[1,2,4] triazole |
| 59 | 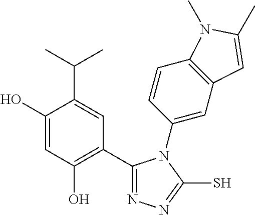 [SMILES: Cc1cc2cc(-n3c(S)nnc3-c3cc(C(C)C)c(O)cc3O)ccc2n1C] | 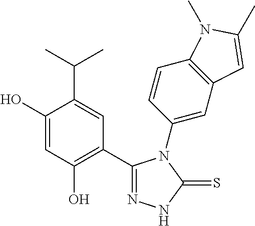 [SMILES: Cc1cc2cc(-n3c(-c4cc(C(C)C)c(O)cc4O)n[nH]c3=S)ccc2n1C] | 3-(2,4-dihydroxy-5- isopropyl-phenyl)-4- (1,2-dimethyl-indol-5- yl)-5-mercapto-[1,2,4] triazole |
| 60 | 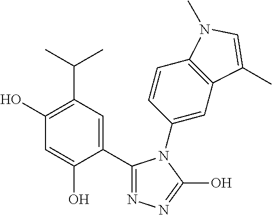 [SMILES: Cc1cn(C)c2ccc(-n3c(O)nnc3-c3cc(C(C)C)c(O)cc3O)cc12] | 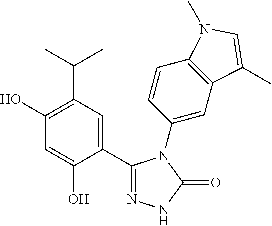 [SMILES: Cc1cn(C)c2ccc(-n3c(-c4cc(C(C)C)c(O)cc4O)n[nH]c3=O)cc12] | 3-(2,4-dihydroxy-5- isopropyl-phenyl)-4- (1,3-dimethyl-indol-5- yl)-5-hydroxy-[1,2,4] triazole |
| 62 | 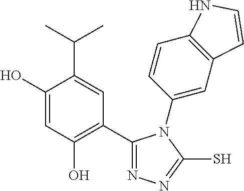 [SMILES: CC(C)c1cc(-c2nnc(S)n2-c2ccc3[nH]ccc3c2)c(O)cc1O] | 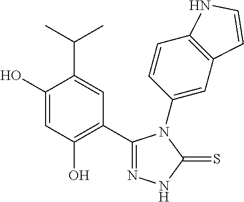 [SMILES: CC(C)c1cc(-c2n[nH]c(=S)n2-c2ccc3[nH]ccc3c2)c(O)cc1O] | 3-(2,4-dihydroxy-5- isopropyl-phenyl)-4- (1H-indol-5-yl)-5- mercapto-[1,2,4] triazole |
| 63 | 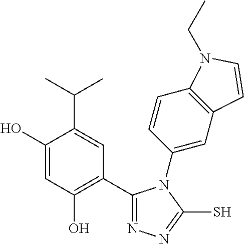 [SMILES: CCn1ccc2cc(-n3c(S)nnc3-c3cc(C(C)C)c(O)cc3O)ccc21] | 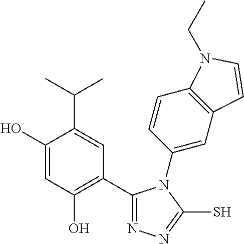 [SMILES: CCn1ccc2cc(-n3c(S)nnc3-c3cc(C(C)C)c(O)cc3O)ccc21] | 3-(2,4-dihydroxy-5- isopropyl-phenyl)-4- (1-ethyl-indol-5-yl)-5- mercapto-[1,2,4] triazole |
| 64 | 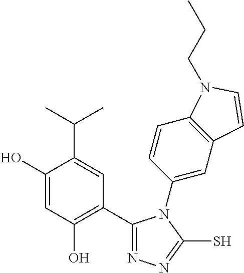 [SMILES: CCCn1ccc2cc(-n3c(S)nnc3-c3cc(C(C)C)c(O)cc3O)ccc21] | 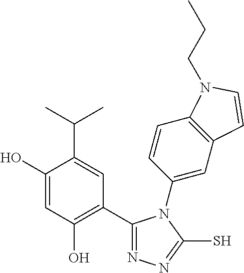 [SMILES: CCCn1ccc2cc(-n3c(S)nnc3-c3cc(C(C)C)c(O)cc3O)ccc21] | 3-(2,4-dihydroxy-5- isopropyl-phenyl)-4- (1-propyl-indol-5-yl)- 5-mercapto-[1,2,4] triazole |
| 65 | 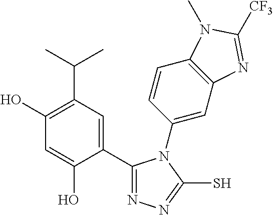 [SMILES: CC(C)c1cc(-c2nnc(S)n2-c2ccc3c(c2)nc(C(F)(F)F)n3C)c(O)cc1O] | 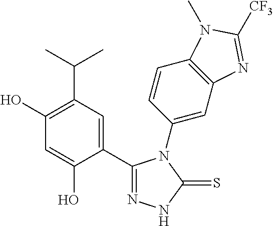 [SMILES: CC(C)c1cc(-c2n[nH]c(=S)n2-c2ccc3c(c2)nc(C(F)(F)F)n3C)c(O)cc1O] | 3-(2,4-dihydroxy-5- isopropyl-phenyl)-4- (1-methyl-2- trifluoromethyl- benzimidazol-5-yl)-5- mercapto-[1,2,4] triazole |
| 66 | 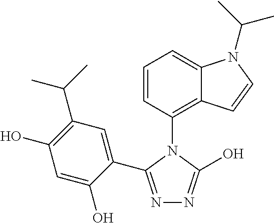 [SMILES: CC(C)c1cc(-c2nnc(O)n2-c2cccc3c2ccn3C(C)C)c(O)cc1O] | 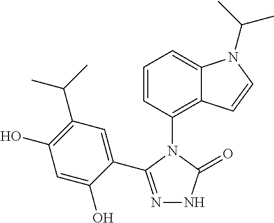 [SMILES: CC(C)c1cc(-c2n[nH]c(=O)n2-c2cccc3c2ccn3C(C)C)c(O)cc1O] | 3-(2,4-dihydroxy-5- isopropyl-phenyl)-4- (1-isopropyl-indol-4- yl)-5-hydroxy-[1,2,4] triazole |

TABLE 2
Compounds according to Formula (Ia)

| No. | Structure | Tautomeric structure | Name |
|---|---|---|---|
| 1a | 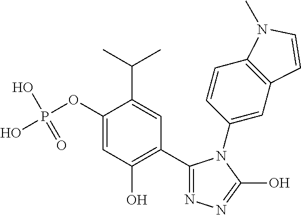 [SMILES: CC(C)c1cc(-c2nnc(O)n2-c2ccc3c(ccn3C)c2)c(O)cc1OP(=O)(O)O] | 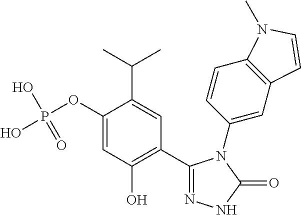 [SMILES: CC(C)c1cc(-c2n[nH]c(=O)n2-c2ccc3c(ccn3C)c2)c(O)cc1OP(=O)(O)O] | 5-hydroxy-4-(5- hydroxy-4-(1- methyl-1H- indol-5-yl)-4H- 1,2,4-triazol-3- yl)-2- isopropylphenyl dihydrogen phosphate |
| 2a | 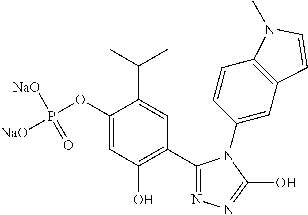 | 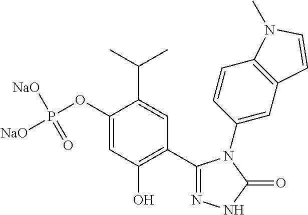 | sodium 5- hydroxy-4-(5- hydroxy-4-(1- methyl-1H- indol-5-yl)-4H- 1,2,4-triazol-3- yl)-2- isopropylphenyl phosphate |
| 3a | 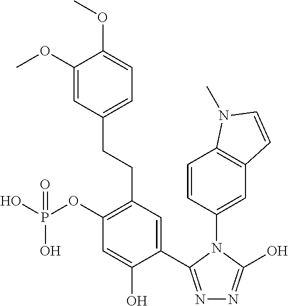 [SMILES: COc1ccc(CCc2cc(-c3nnc(O)n3-c3ccc4c(ccn4C)c3)c(O)cc2OP(=O)(O)O)cc1OC] | 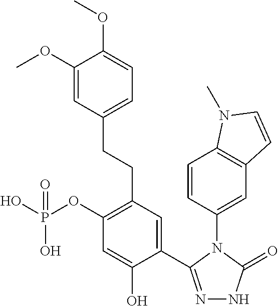 [SMILES: COc1ccc(CCc2cc(-c3n[nH]c(=O)n3-c3ccc4c(ccn4C)c3)c(O)cc2OP(=O)(O)O)cc1OC] | 2-(3,4- dimethoxyphenethyl)- 5-hydroxy- 4-(5-hydroxy-4- (1-methyl-1H- indol-5-yl)-4H- 1,2,4-triazol-3- yl)phenyl dihydrogen phosphate |
| 4a | 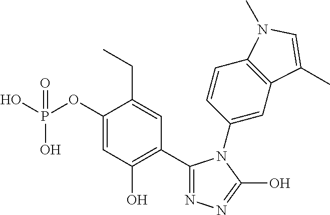 [SMILES: CCc1cc(-c2nnc(O)n2-c2ccc3c(c2)c(C)cn3C)c(O)cc1OP(=O)(O)O] | 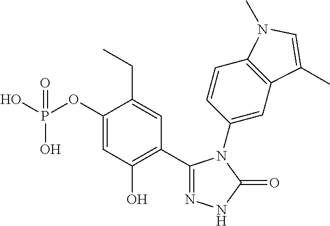 [SMILES: CCc1cc(-c2n[nH]c(=O)n2-c2ccc3c(c2)c(C)cn3C)c(O)cc1OP(=O)(O)O] | 4-(4-(1,3- dimethyl-1H- indol-5-yl)-5- hydroxy-4H- 1,2,4-triazol-3- yl)-2-ethyl-5- hydroxyphenyl dihydrogen phosphate |

Compounds used in the disclosed methods can be prepared according to methods disclosed in U.S. Application No. 2006-0167070 and WO2009/023211.

The triazolone compounds described herein typically can form a tautomeric structure as shown below and as exemplified by the tautomeric structures shown in Tables 1 and 2:

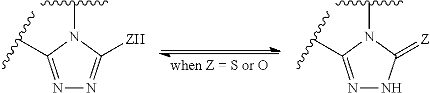

Methods of treating, managing, or ameliorating cancer, or one or more symptoms thereof, include administering to a subject in need thereof one or more compounds represented by the structural formulae (I) or (Ia) or a compound in Table 1 or Table 2, in combination with radiotherapy, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, lung cancer and multiple myeloma.

In one embodiment, a method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy. In one embodiment, the triazolone compound is administered synergistically with the radiotherapy for the treatment of cancer. In one embodiment, the synergistic treatment is for cervical cancer. In one embodiment, the synergistic treatment is for lung cancer. In one embodiment, the synergistic treatment is for breast cancer. In one embodiment, the synergistic treatment is for colon cancer. In one embodiment, the synergistic treatment is for non-small cell lung cancer. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg.

In one embodiment, a method of treating a subject with cancer includes administering to the subject a synergistic amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy. In one embodiment, the synergistic amount of the triazolone compound is within the range from about 0.15 mg/kg to about 1000 mg/kg. In one embodiment, the synergistic amount of the triazolone compound is within the range from about 1 mg/day to about 100 g/day. In one embodiment, the synergistic amount of the triazolone compound is within the range from about 100 mg/day to about 1000 mg/day. In one embodiment, the synergistic amount of the triazolone compound is within the range from about 100 mg to about 1000 mg. In one embodiment, the treatment is for a subject with cervical cancer. In one embodiment, the treatment is for a subject with lung cancer. In one embodiment, the treatment is for a subject with breast cancer. In one embodiment, the treatment is for a subject with colon cancer. In one embodiment, the treatment is for a subject with non-small cell lung cancer.

In one embodiment, a method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy. In one embodiment, the triazolone compound is administered synergistically with the radiotherapy for the treatment of cancer. In one embodiment, the synergistic treatment is for cervical cancer. In one embodiment, the synergistic treatment is for lung cancer. In one embodiment, the synergistic treatment is for breast cancer. In one embodiment, the synergistic treatment is for colon cancer. In one embodiment, the synergistic treatment is for non-small cell lung cancer. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg.

In one embodiment, a method of treating a subject with cancer includes administering to the subject a synergistic amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy. In one embodiment, the synergistic amount of the triazolone compound is within the range from about 0.15 mg/kg to about 1000 mg/kg. In one embodiment, the synergistic amount of the triazolone compound is within the range from about 1 mg/day to about 100 g/day. In one embodiment, the synergistic of the triazolone compound is within the range from about 100 mg/day to about 1000 mg/day. In one embodiment, the synergistic of the triazolone compound is within the range from about 100 mg to about 1000 mg. In one embodiment, the treatment is for a subject with cervical cancer. In one embodiment, the treatment is for a subject with lung cancer. In one embodiment, the treatment is for a subject with breast cancer. In one embodiment, the treatment is for a subject with colon cancer. In one embodiment, the treatment is for a subject with non-small cell lung cancer.

In one embodiment, a method of treating a subject with cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In one embodiment, a method of treating a subject with cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In one embodiment, a method of treating a subject with cancer, wherein the subject is being or has been treated with a chemotherapeutic agent, includes administering to the subject an effective amount of a triazolone compound represented by the structural formulae (I) or (Ia) or a compound in Table 1 or Table 2, in combination with radiotherapy.

In one embodiment, a method of treating a subject with cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma, and the subject is being or has been treated with a chemotherapeutic agent, includes administering to the subject an effective amount of a triazolone compound represented by the structural formulae (I) or (Ia) or a compound in Table 1 or Table 2, in combination with radiotherapy. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In one embodiment, a method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy.

In one embodiment, a method of treating a subject with cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma, and the subject is being or has been treated with a chemotherapeutic agent, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In one embodiment, a method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy.

In one embodiment, a method of treating a subject with cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma, and the subject is being or has been treated with a chemotherapeutic agent, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In another embodiment, a method of potentiating or improving radiotherapy cancer treatment includes administering to a patient in need thereof a therapeutically effective amount of a triazolone compound represented by the structural formulae (I) or (Ia) or a compound in Table 1 or Table 2, and directing radiotherapy at a prescribed dosage to a locus of cancer.

In another embodiment, a method of potentiating or improving radiotherapy cancer treatment includes administering to a patient in need thereof a therapeutically effective amount of a triazolone compound represented by the structural formulae (I) or (Ia) or a compound in Table 1 or Table 2, and directing radiotherapy at a prescribed dosage to a locus of cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In another embodiment, a method of potentiating or improving radiotherapy cancer treatment includes administering to a patient in need thereof a therapeutically effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, and directing radiotherapy at a prescribed dosage to a locus of cancer.

In another embodiment, a method of potentiating or improving radiotherapy cancer treatment includes administering to a patient in need thereof a therapeutically effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, and directing radiotherapy at a prescribed dosage to a locus of cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the cancer is cervical cancer. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg. In one embodiment, the cancer is lung cancer. In one embodiment, the cancer is breast cancer. In one embodiment, the cancer is colon cancer. In one embodiment, the cancer is non-small cell lung cancer.

In another embodiment, a method of potentiating or improving radiotherapy cancer treatment includes administering to a patient in need thereof a therapeutically effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and directing radiotherapy at a prescribed dosage to a locus of cancer.

In another embodiment, a method of potentiating or improving radiotherapy cancer treatment includes administering to a patient in need thereof a therapeutically effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and directing radiotherapy at a prescribed dosage to a locus of cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In yet another embodiment, a method of enhancing the cytotoxic effects of radiation therapy includes administering to a subject in need of said radiation therapy a therapeutically effective amount of a triazolone compound represented by the structural formulae (I) or (Ia) or a compound in Table 1 or Table 2, or a tautomer, or a pharmaceutically acceptable salt thereof, in combination with the administration of a dosage of the radiation therapy, whereby the cytotoxic effect of said radiation therapy is increased compared to that which would occur in the absence of the triazolone compound; wherein the dosage of said radiation therapy is decreased as compared to a dosage effective without administering the triazolone compound, and wherein said subject is afflicted with cancer.

In yet another embodiment, a method of enhancing the cytotoxic effects of radiation therapy includes administering to a subject in need of said radiation therapy a therapeutically effective amount of a triazolone compound represented by the structural formulae (I) or (Ia) or a compound in Table 1 or Table 2, or a tautomer, or a pharmaceutically acceptable salt thereof, in combination with the administration of a dosage of the radiation therapy, whereby the cytotoxic effect of said radiation therapy is increased compared to that which would occur in the absence of the triazolone compound; wherein the dosage of said radiation therapy is decreased as compared to a dosage effective without administering the triazolone compound, and wherein said subject is afflicted with cancer selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg. In one embodiment, the cancer is cervical cancer. In one embodiment, the cancer is lung cancer. In one embodiment, the cancer is breast cancer. In one embodiment, the cancer is colon cancer.

In yet another embodiment, a method of enhancing the cytotoxic effects of radiation therapy includes administering to a subject in need of said radiation therapy a therapeutically effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with the administration of a dosage of the radiation therapy, whereby the cytotoxic effect of said radiation therapy is increased compared to that which would occur in the absence of the triazolone compound; wherein the dosage of said radiation therapy is decreased as compared to a dosage effective without administering the triazolone compound, and wherein said subject is afflicted with cancer.

In yet another embodiment, a method of enhancing the cytotoxic effects of radiation therapy includes administering to a subject in need of said radiation therapy a therapeutically effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with the administration of a dosage of the radiation therapy, whereby the cytotoxic effect of said radiation therapy is increased compared to that which would occur in the absence of the triazolone compound; wherein the dosage of said radiation therapy is decreased as compared to a dosage effective without administering the triazolone compound, and wherein said subject is afflicted with cancer selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In yet another embodiment, a method of enhancing the cytotoxic effects of radiation therapy includes administering to a subject in need of said radiation therapy a therapeutically effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with the administration of a dosage of the radiation therapy, whereby the cytotoxic effect of said radiation therapy is increased compared to that which would occur in the absence of the triazolone compound; wherein the dosage of said radiation therapy is decreased as compared to a dosage effective without administering the triazolone compound, and wherein said subject is afflicted with cancer.

In yet another embodiment, a method of enhancing the cytotoxic effects of radiation therapy includes administering to a subject in need of said radiation therapy a therapeutically effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a pharmaceutically acceptable salt thereof, in combination with the administration of a dosage of the radiation therapy, whereby the cytotoxic effect of said radiation therapy is increased compared to that which would occur in the absence of the triazolone compound; wherein the dosage of said radiation therapy is decreased as compared to a dosage effective without administering the triazolone compound, and wherein said subject is afflicted with cancer selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the effective amount of the triazolone compound is within the range from about 0.15 mg/kg to about 1000 mg/kg. In one embodiment, the effective amount of the triazolone compound is within the range from about 1 mg/day to about 100 g/day. In one embodiment, the effective amount of the triazolone compound is within the range from about 100 mg/day to about 1000 mg/day. In one embodiment, the effective amount of the triazolone compound is within the range from about 100 mg to about 1000 mg. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In yet another embodiment, a method of sensitizing tumor or cancer cells to radiotherapy includes administering to a subject afflicted with cancer a therapeutically effective amount of a triazolone compound represented by the structural formulae (I) or (Ia) or a compound in Table 1 or Table 2, or a tautomer, or a pharmaceutically acceptable salt thereof, in combination with radiotherapy.

In yet another embodiment, a method of sensitizing tumor or cancer cells to radiation therapy includes administering to a subject afflicted with cancer a therapeutically effective amount of a triazolone compound represented by the structural formulae (I) or (Ia) or a compound in Table 1 or Table 2, or a tautomer, or a pharmaceutically acceptable salt thereof, in combination with radiotherapy, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In yet another embodiment, a method of sensitizing tumor or cancer cells to radiation therapy includes administering to a subject afflicted with cancer a therapeutically effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy.

In yet another embodiment, a method of sensitizing tumor or cancer cells to radiation therapy includes administering to a subject afflicted with cancer a therapeutically effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with radiotherapy, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In yet another embodiment, a method of sensitizing tumor or cancer cells to radiation therapy includes administering to a subject afflicted with cancer a therapeutically effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy.

In yet another embodiment, a method of sensitizing tumor or cancer cells to radiation therapy includes administering to a subject afflicted with cancer a therapeutically effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a pharmaceutically acceptable salt thereof, in combination with radiotherapy, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma. In one embodiment, the cancer is cervical cancer. In one embodiment, the cancer is lung cancer. In one embodiment, the cancer is breast cancer. In one embodiment, the cancer is colon cancer. In one embodiment, the cancer is non-small cell lung cancer.

In another embodiment, a method of inhibiting the growth of a cell includes the steps of: (a) first contacting said cell with a compound of formulae (I) or (Ia) or a compound in Table (1) or Table (2), or tautomer or a pharmaceutically acceptable salt thereof in an amount effective to sensitize said cell to ionizing radiation; and then (b) exposing said cell to a dose of ionizing radiation effective to inhibit the growth of said cell.

In another embodiment, a method of inhibiting the growth of a cell comprising the steps of: (a) first contacting said cell with a compound of 3-(2,4-dihydroxy-5-isopropyl-phenyl)-4-(1-methyl-indol-5-yl)-5-hydroxy-[1,2,4]triazole, or tautomer or a pharmaceutically acceptable salt thereof in an amount effective to sensitize said cell to ionizing radiation; and then (b) exposing said cell to a dose of ionizing radiation effective to inhibit the growth of said cell.

The therapeutic agents of the combination therapies of the invention can be administered sequentially or concurrently. In one embodiment, the administration of the Hsp90 inhibitor and radiotherapy are done concurrently. In another embodiment, the administration of the Hsp90 inhibitor and radiotherapy are done separately. In another embodiment, the administration of the Hsp90 inhibitor and radiotherapy are done sequentially. In another embodiment, the administration of the Hsp90 inhibitor is administered prior to or after radiotherapy. In one embodiment, the administration of the HSP90 inhibitor and radiotherapy are done until the cancer is cured or stabilized or improved.

In a specific embodiment, the combination therapies of the invention comprise one or more compounds and at least one other therapy which has the same mechanism of action as said compounds. In another specific embodiment, the combination therapies of the invention comprise one or more compounds of the invention and at least one other therapy which has a different mechanism of action than said compounds. In certain embodiments, the combination therapies of the present invention improve the therapeutic effect of one or more triazolone compounds described herein by functioning together with the radiotherapy to have an additive or synergistic effect. In certain embodiments, the combination therapies of the present invention reduce the side effects associated with the therapies. In certain embodiments, the combination therapies of the present invention reduce the effective dosage of one or more of the therapies.

In a specific embodiment, a pharmaceutical composition comprising one or more triazolone compounds described herein is administered to a subject, preferably a human, to prevent, treat, manage, or ameliorate cancer, or one or more symptom thereof. In accordance with the invention, pharmaceutical compositions of the invention may also comprise one or more other agents being used, have been used, or are known to be useful in the treatment or amelioration of cancer, particularly colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma.

Methods for managing, treating or ameliorating cancer, particularly colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma, or one or more symptoms thereof in a subject refractory (either completely or partially) to existing agent therapies for cancer, comprise administering to a subject a dose of an effective amount of one or more triazolone compounds described herein and a dose of radiotherapy. Methods for treating, managing, or ameliorating cancer, particularly colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, small cell lung carcinoma, non-small cell lung cancer, and multiple myeloma, or a symptom thereof comprise administering one or more compounds of the invention in combination with radiotherapy to patients who have proven refractory to other therapies but are no longer on these therapies.

The triazolone compounds described herein can be administered to a subject by any route known to one of skill in the art. Examples of routes of administration include parenteral, e.g., intravenous, intradermal, subcutaneous, oral (e.g., inhalation), intranasal, transdermal (topical), transmucosal, and rectal administration.

The present invention is useful for the treatment, and amelioration of cancer, particularly relapsed or refractory cancer. In a specific embodiment, a composition comprising one or more triazonlone compounds described herein, or a pharmaceutically acceptable salt thereof, is administered in combination with radiotherapy. In another embodiment, a composition comprising one or more therapeutic agents other than a triazolone compound described herein, or a pharmaceutically acceptable salt, is administered in combination with radiotherapy. In another embodiment, a composition comprising one or more triazolone compounds described herein, or a pharmaceutically acceptable salt thereof, and one or more other therapeutic agents is administered, in combination with radiotherapy. Pharmaceutical compositions used herein are formulated to be compatible with its intended route of administration. Examples of routes of administration include parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings.

Typical pharmaceutical compositions and dosage forms comprise one or more excipients. Suitable excipients are well known to those skilled in the art of pharmacy.

The triazolone compounds described herein can be also formulated into or administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566,

In general, the recommended daily dose range of a triazolone compound for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different cancers, as will be readily known by those of ordinary skill in the art. Similarly, amounts sufficient to prevent, manage, treat or ameliorate such cancers, but insufficient to cause, or sufficient to reduce, adverse effects associated with the triazolone compounds described herein are also encompassed by the above described dosage amounts and dose frequency schedules. Further, when a patient is administered multiple dosages of a triazolone compound described herein, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition comprising a triazolone compound described herein administered to prevent, treat, manage, or ameliorate cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition comprising a compound described herein administered to prevent, treat, manage, or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 of 7 days, or once weekly, once every two weeks, once every three weeks or once monthly.

In certain embodiments, when the triazolone compounds described herein are administered in combination with radiotherapy, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies are administered within the same patent visit.

In certain embodiments, one or more compounds describer herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, a method of preventing, treating, managing, or ameliorating a proliferative disorders, such as cancer, or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

### EXAMPLES

Compound 1 Enhances the Anti-Tumor Activity of Ionizing Radiation Against Human Tumor Cells in a Mouse Xenograft Model

The human cervical carcinoma cancer cell line, HeLaHL, carrying a human Hsp70 promoter-luciferase reporter construct, was obtained from the laboratory of Dr. Richard Morimoto (Northwestern University, Evanston, Ill., USA). The cells were cultured in Dulbecco's Modified Eagle growth media prepared with 10% fetal bovine serum (FBS), 1% 100×HEPES, 1% 100× Penicillin-Streptomycin, 1% 100×L-glutamine, 1% 100× sodium pyruvate and 1% 100×MEM non-essential amino acids. FBS was obtained from American Type Culture Collection (Manassas, Va., USA) and all other reagents were obtained from Invitrogen Corp. (Carlsbad, Calif., USA). Cells that had been cryopreserved in liquid nitrogen were rapidly thawed at 37° C. and transferred to a tissue culture flask containing growth media and then incubated at 37° C. in a 5% CO₂ incubator. To expand the cell line, cultures were passaged every 3 to 4 days by washing with 10 mL of room temperature phosphate buffered saline (PBS) and then disassociating cells by adding 5 mL 1× trypsin-EDTA until the cells detached from the surface of the flask. To inactivate the trypsin, 10 mL of growth media was added and then the contents of the flask were seeded 1:4 into 175 cm² flasks containing 50 mL of growth media and incubated at 37° C. in a 5% CO₂ incubator. When the flasks reached 90% confluence, the above passaging process was repeated until sufficient cells had been obtained for implantation into mice.

Eight week old, female homozygous Crl:CD1-Foxn1^{nu/nu} (Nude) mice were obtained from Charles River Laboratories (Wilmington, Mass., USA). Animals were housed 4-5/cage in micro-isolators, with a 12 hr/12 hr light/dark cycle, acclimated for at least 1 week prior to use and fed normal laboratory chow ad libitum. Studies were conducted on animals at 10 weeks of age at implantation. To implant HeLaHL tumor cells into Nude mice, cell cultures were trypsinized as above and centrifuged to pellet the cells. The supernatant was aspirated and the cell pellet was resuspended at a concentration of 1×10⁸ cells/mL in non-supplemented media. Cell concentration was determined using a hemocytometer. Using a 27 gauge needle and 1 cc syringe, 0.1 mL of the cell suspension was injected subcutaneously into the flanks of Nude mice. Tumor volumes (V) were calculated by caliper measurement of the width (W), length (L) and thickness (T) of tumors using the following formula: V=0.5236×(L×W×T).

HeLaHL tumors were then permitted to develop in vivo until the majority reached 90-240 mm³ in tumor volume, which required approximately 2½ weeks following implantation. Animals with oblong, very small or large tumors were discarded, and only animals carrying tumors that displayed consistent growth rates were selected for studies. Animals were randomized into treatment groups so that the average tumor volumes of each group were similar at the start of dosing. % T/C values, as a measure of efficacy, were determined as follows:
(i) If ΔT>0: % T/C=(ΔT/ΔC)×100
(ii) If ΔT<0: % T/C=(ΔT/T₀)×100
(iii) ΔT=Change in average tumor volume between start of dosing and the end of study.
(iv) ΔC=Change in average tumor volume between start of dosing and the end of study.
(v) T₀=Average tumor volume at start of dosing.

To formulate Compound 1 in 10/18 DRD, stock solutions of Compound 1 were prepared by dissolving the appropriate amounts of the compound in dimethyl sulfoxide (DMSO) by sonication in an ultrasonic water bath. Stock solutions were prepared weekly, stored at −20° C. and diluted fresh each day for dosing. A solution of 20% Cremophor RH40 (polyoxyl 40 hydrogenated castor oil; BASF Corp., Aktiengesellschaft, Ludwigshafen, Germany) in 5% dextrose in water (D5W; Abbott Laboratories, North Chicago, Ill., USA) was also prepared by first heating 100% Cremophor RH40 at 50-60° C. until liquefied and clear, diluting 1:5 with 100% D5W, reheating again until clear and then mixing well. This solution was stored at room temperature for up to 3 months prior to use. To prepare 10/18 DRD formulations for daily dosing, DMSO stock solutions were diluted 1:10 with 20% Cremophor RH40. The final 10/18 DRD formulation for dosing contained 10% DMSO, 18% Cremophor RH40, 3.6% dextrose, 68.4% water and the appropriate amount of test article. Animals were intravenously (i.v.) injected with this formulation at 10 mL per kg body weight on one day each week (on the same day with one of the 3 weekly radiation doses) for a total of 4 doses.

Animals were irradiated using a calibrated ¹³⁷Cs gamma-ray (0.662 MeV) Model 30 Mark I irradiator (J. L. Shepherd & Associates, San Fernando, Calif., USA). Tumor-bearing animals were anesthetized for approximately 4 minutes with inhaled isoflurane (IsoFlo, Abbott Laboratories, North Chicago, Ill., USA) delivered on a stream of oxygen at 0.8 L/min and 3% volume/volume on a mobile anesthesia machine (VetEquip, Pleasanton, Calif., USA). This resulted in sufficient anesthesia to immobilize the animals for placement of each group of 4-5 animals into the irradiator, but the effect typically wore off 1-2 minutes prior to completion of irradiation for that group. Anesthetized animals were positioned in the irradiator approximately 2.5 cm in front of the ¹³⁷Cs source using a restraint device capable of holding 5 animals (J. L Shepherd & Associates). Ionizing radiation was focused on the subcutaneously implanted HeLaHL tumors through a 1 cm wide collimator (J. L. Shepherd & Associates) placed in front of the ¹³⁷Cs source Animals were irradiated with 2 Gy (200 rad) on a 3×/week schedule for 3 weeks. Dosimetry demonstrated an approximately 450:1 ratio of focused radiation received by tumors relative to the head and shoulder region of animals.

### Example 1

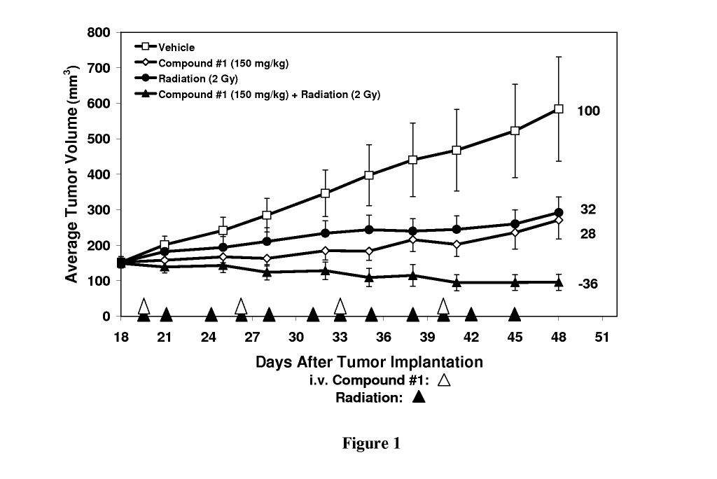

The ability of Compound 1 to enhance the in vivo anti-tumor activity of ionizing radiation was investigated. Tumor-bearing animals (8 mice/group) were i.v. injected 1 time per week for a total of 4 doses (open arrowheads) with 10 mL/kg of 10/18 DRD vehicle with or without 150 mg/kg Compound 1. Animals were also irradiated 3 times per week for a total of 12 doses (closed arrowheads) with 2 Gy radiation. In the combination treatment group, animals were dosed with Compound 15 hr before being irradiated on days on which both treatments were delivered together. Each group that was not dosed with Compound 1 was instead dosed with 10/18 DRD vehicle as a mock treatment. Each group that was not irradiated was instead anesthetized and restrained as a mock irradiation. Average tumor volumes for each group were determined every 3-5 days (error bars represent +/−SEM). Treatment with a combination of 2 Gy radiation, delivered three times per week, in combination with 150 mg/kg Compound 1, dosed one time per week, induced substantial tumor regression relative to that achieved by either single therapy alone. % T/C values are indicated on the right. Systemic toxicity was observed in the Compound 1 plus 2 Gy radiation treatment group. The average bodyweight changes relative to the start of the study for all treatment groups was between 0.4% and 3.2% over the course of the study. As shown in FIG. 1, treatment with either 150 mg/kg Compound 1 dosed 1 time per week, or irradiation with 2 Gy ionizing radiation delivered 3 times per week, resulted in moderate reductions in the growth rate of HeLaHL cells in Nude mice, with % T/C values of 28 and 32, respectively. However, treatment with 2 Gy radiation delivered 3 times per week, combined with 150 mg/kg Compound 1 dosed 1 time per week (Compound 1 was dosed 5 hr before irradiation on days where both treatments were delivered), resulted in substantial tumor regression of HeLaHL cells in Nude mice, with a % T/C value of −36. However, this effect was associated with toxicity on Day 42 of the study. 2/8 animals receiving treatment with 2 Gy radiation combined with 150 mg/kg Compound 1 were removed from the study due to >20% body weight loss. One of the animals removed later died on Day 45 of the study due to excessive body weight loss. Over the course of the study, the vehicle, Compound 1 alone, radiation alone, and Compound 1 plus radiation treatment groups had average bodyweight changes relative to the start of the study of 0.7%, 3.2%, 1.3% and 0.4%, respectively. It can be seen from FIG. 1 that Compound 1, in combination with radiotherapy of 2 Gy radiation, induced substantial tumor regression relative to that achieved by either therapy alone.

### Example 2

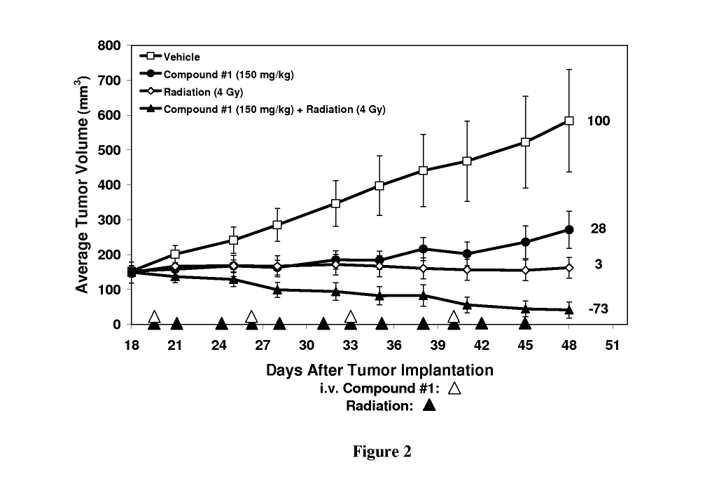

Tumor-bearing animals (8 mice/group) were i.v. injected 1 time per week for a total of 4 doses (open arrowheads) with 10 mL/kg of 10/18 DRD vehicle with or without 150 mg/kg Compound 1. Animals were also irradiated 3 times per week for a total of 12 doses (closed arrowheads) with 3-4 Gy radiation. The dose of 4 Gy radiation was reduced to 3 Gy radiation on Day 40 due to systemic toxicity. In the combination treatment group, animals were dosed with Compound 15 hr before being irradiated on days on which both treatments were delivered together. Each group that was not dosed with Compound 1 was instead dosed with 10/18 DRD vehicle as a mock treatment. Each group that was not irradiated was instead anesthetized and restrained as a mock irradiation. Average tumor volumes for each group were determined every 3-5 days (error bars represent +/−SEM). Treatment with a combination of 3-4 Gy radiation, delivered three times per week, in combination with 150 mg/kg Compound 1, dosed one time per week, induced significant tumor regression relative to that achieved by either single therapy alone. % T/C values are indicated on the right. Systemic toxicity was observed in the Compound 1 plus 4 Gy radiation treatment group. The average bodyweight changes relative to the start of the study for all treatment groups was between 0.7% and 3.2% over the course of the study. As shown in FIG. 2, treatment with 150 mg/kg Compound 1 dosed 1 time per week resulted in a moderate reduction in the growth rate of HeLaHL cells in Nude mice, with a % T/C value of 28, while irradiation with 3-4 Gy ionizing radiation delivered 3 timers per week, resulted in a more substantial reduction in the growth rate with a % T/C value of 3. However, treatment with 3-4 Gy radiation delivered 3 times per week, combined with 150 mg/kg Compound 1 dosed 1 time per week (Compound 1 was dosed 5 hr before irradiation on days where both treatments were delivered), resulted in significant tumor regression of HeLaHL cells in Nude mice, with a % T/C value of −73. However, this effect was associated with toxicity on Day 34 of the study. 1/8 animals receiving treatment with 4 Gy radiation combined with 150 mg/kg Compound 1 was removed from the study due to >20% body weight loss. The radiation dose was reduced on Day 40 of the study from 4 Gy to 3 Gy. Over the course of the study, the vehicle, Compound 1 alone, radiation alone, and Compound 1 plus radiation treatment groups had average bodyweight changes relative to the start of the study of 0.7%, 3.2%, 2.8% and 1.2%, respectively. It can be seen from FIG. 2 that Compound 1, in combination with radiotherapy of 4 Gy radiation, induced significantly tumor regression relative to that achieved by either therapy alone.

### Example 3

Cell viability assays are implemented to assess the effective concentrations of Compound 1 in head and neck cancer tumor cell growth in vitro. This is accomplished by plating head and neck cancer cells at a pre-determined optimal seeding density and dosing with a serial dilution of Compound 1 or vehicle. Cell viability is determined 24 hr later by the alamarBlue assay (Invitrogen). On the basis of the cytotoxicity data, head and neck cancer cells are exposed to Compound 1 for 24 hr, followed by X-ray exposure from 0-8 Gy. Radiation sensitivity is subsequently analyzed by the colony survival test as previously described, see, e.g. Stingl et al., Novel HSP90 inhibitors, NVP-AUY922 and NVP-BEP800, radiosensitise tumour cells through cell-cycle impairment, increased DNA damage and repair protraction. Br J Cancer. 102(11): p. 1578-91. To validate the radiosensitizing effects of Compound 1, xenograft model is utilized. Head and neck cancer cells used in vitro are grown as tumor xenografts in nude mice. Head and neck tumor is permitted to develop in vivo until the majority reaches 90-240 mm³. Animals are then randomized into treatment groups so that the average tumor volumes of each group are similar at the start of dosing. Animals are then dosed by intravenous injections one time per week for a total of four doses with vehicle of Compound 1 (50 or 150 mg/kg, 1×/week). Animals are also irradiated three times per week for a total of twelve doses (0-4 Gy radiation). In the combination treatment groups, animals are dosed with Compound 1 five hours prior to irradiation. Average tumor volumes and body weights for each group are determined every 3-5 days.

### Example 4

Cell viability assays are implemented to assess the effective concentrations of Compound 1 in colorectal cancer tumor cell growth in vitro. This is accomplished by plating colorectal cancer cells at a pre-determined optimal seeding density and dosing with a serial dilution of Compound 1 or vehicle. Cell viability is determined 24 hr later by the alamarBlue assay (Invitrogen). On the basis of the cytotoxicity data, colorectal cancer cells are exposed to Compound 1 for 24 hr, followed by X-ray exposure from 0-8 Gy. Radiation sensitivity is subsequently analyzed by the colony survival test as previously described. To validate the radiosensitizing effects of Compound 1, xenograft model is utilized. Colorectal cancer cells used in vitro are grown as tumor xenografts in nude mice. Colorectal tumor is permitted to develop in vivo until the majority reaches 90-240 mm³. Animals are then randomized into treatment groups so that the average tumor volumes of each group are similar at the start of dosing. Animals are then dosed by intravenous injections one time per week for a total of four doses with vehicle of Compound 1 (50 or 150 mg/kg, 1×/week) Animals are also irradiated three times per week for a total of twelve doses (0-4 Gy radiation). In the combination treatment groups, animals are dosed with Compound 1 five hours prior to irradiation. Average tumor volumes and body weights for each group are determined every 3-5 days.

### Example 5

Cell viability assays are implemented to assess the effective concentrations of Compound 1 in pancreatic cancer tumor cell growth in vitro. This is accomplished by plating pancreatic cancer cells at a pre-determined optimal seeding density and dosing with a serial dilution of Compound 1 or vehicle. Cell viability is determined 24 hr later by the alamarBlue assay (Invitrogen). On the basis of the cytotoxicity data, pancreatic cancer cells are exposed to Compound 1 for 24 hr, followed by X-ray exposure from 0-8 Gy. Radiation sensitivity is subsequently analyzed by the colony survival test as previously described. To validate the radiosensitizing effects of Compound 1, xenograft model is utilized. Pancreatic cancer cells used in vitro are grown as tumor xenografts in nude mice. Pancreatic tumor is permitted to develop in vivo until the majority reaches 90-240 mm³. Animals are then randomized into treatment groups so that the average tumor volumes of each group are similar at the start of dosing. Animals are then dosed by intravenous injections one time per week for a total of four doses with vehicle of Compound 1 (50 or 150 mg/kg, 1×/week) Animals are also irradiated three times per week for a total of twelve doses (0-4 Gy radiation). In the combination treatment groups, animals are dosed with Compound 1 five hours prior to irradiation. Average tumor volumes and body weights for each group are determined every 3-5 days.

### Example 6

Cell viability assays are implemented to assess the effective concentrations of Compound 1 in breast cancer tumor cell growth in vitro. This is accomplished by plating breast cancer cells at a pre-determined optimal seeding density and dosing with a serial dilution of Compound 1 or vehicle. Cell viability is determined 24 hr later by the alamarBlue assay (Invitrogen). On the basis of the cytotoxicity data, breast cancer cells are exposed to Compound 1 for 24 hr, followed by X-ray exposure from 0-8 Gy. Radiation sensitivity is subsequently analyzed by the colony survival test as previously described. To validate the radiosensitizing effects of Compound 1, xenograft model is utilized. Breast cancer cells used in vitro are grown as tumor xenografts in nude mice. Breast cancer is permitted to develop in vivo until the majority reaches 90-240 mm³. Animals are then randomized into treatment groups so that the average tumor volumes of each group are similar at the start of dosing. Animals are then dosed by intravenous injections one time per week for a total of four doses with vehicle of Compound 1 (50 or 150 mg/kg, 1×/week) Animals are also irradiated three times per week for a total of twelve doses (0-4 Gy radiation). In the combination treatment groups, animals are dosed with Compound 1 five hours prior to irradiation. Average tumor volumes and body weights for each group are determined every 3-5 days.

### Example 7

Cell viability assays are implemented to assess the effective concentrations of Compound 1 in prostate cancer tumor cell growth in vitro. This is accomplished by plating prostate cancer cells at a pre-determined optimal seeding density and dosing with a serial dilution of Compound 1 or vehicle. Cell viability is determined 24 hr later by the alamarBlue assay (Invitrogen). On the basis of the cytotoxicity data, prostate cancer cells are exposed to Compound 1 for 24 hr, followed by X-ray exposure from 0-8 Gy. Radiation sensitivity is subsequently analyzed by the colony survival test as previously described. To validate the radiosensitizing effects of Compound 1, xenograft model is utilized. Prostate cancer cells used in vitro are grown as tumor xenografts in nude mice. Prostate cancer is permitted to develop in vivo until the majority reaches 90-240 mm³. Animals are then randomized into treatment groups so that the average tumor volumes of each group are similar at the start of dosing. Animals are then dosed by intravenous injections one time per week for a total of four doses with vehicle of Compound 1 (50 or 150 mg/kg, 1×/week) Animals are also irradiated three times per week for a total of twelve doses (0-4 Gy radiation). In the combination treatment groups, animals are dosed with Compound 1 five hours prior to irradiation. Average tumor volumes and body weights for each group are determined every 3-5 days.

### Example 8

Cell viability assays are implemented to assess the effective concentrations of Compound 1 in colon cancer tumor cell growth in vitro. This is accomplished by plating colon cancer cells at a pre-determined optimal seeding density and dosing with a serial dilution of Compound 1 or vehicle. Cell viability is determined 24 hr later by the alamarBlue assay (Invitrogen). On the basis of the cytotoxicity data, colon cancer cells are exposed to Compound 1 for 24 hr, followed by X-ray exposure from 0-8 Gy. Radiation sensitivity is subsequently analyzed by the colony survival test as previously described. To validate the radiosensitizing effects of Compound 1, xenograft model is utilized. Colon cancer cells used in vitro are grown as tumor xenografts in nude mice. Colon cancer is permitted to develop in vivo until the majority reaches 90-240 mm³. Animals are then randomized into treatment groups so that the average tumor volumes of each group are similar at the start of dosing. Animals are then dosed by intravenous injections one time per week for a total of four doses with vehicle of Compound 1 (50 or 150 mg/kg, 1×/week) Animals are also irradiated three times per week for a total of twelve doses (0-4 Gy radiation). In the combination treatment groups, animals are dosed with Compound 1 five hours prior to irradiation. Average tumor volumes and body weights for each group are determined every 3-5 days.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

What is claimed is:

1-23. (canceled)

24. A method of treating cancer, comprising administering to a subject an effective amount of a triazolone compound of 3-(2,4-dihydroxy-5-isopropyl-phenyl)-4-(1-methyl-indol-5-yl)-5-hydroxy-[1,2,4]triazole, or 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with radiotherapy, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, lung cancer, small cell lung carcinoma, non-small cell lung cancer, and multiple myeloma.

25. The method of claim 24, wherein the cancer is colon carcinoma, breast cancer, cervical cancer, lung cancer, small cell lung carcinoma, or non-small cell lung cancer.

26. A method of enhancing the cytotoxic effects of radiation therapy, comprising administering to a subject in need of said radiation therapy a therapeutically effective amount of a triazolone compound of 3-(2,4-dihydroxy-5-isopropyl-phenyl)-4-(1-methyl-indol-5-yl)-5-hydroxy-[1,2,4]triazole, or 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with the administration of a dosage of the radiation therapy, whereby the cytotoxic effect of said radiation therapy is increased compared to that which would occur in the absence of the triazolone compound; wherein the dosage of said radiation therapy is decreased as compared to a dosage effective without administering the triazolone compound, and wherein said subject is afflicted with colon carcinoma, colorectal cancer, gastric cancer, hepatocellular cancer, pancreatic cancer, breast cancer, ovarian cancer, prostate cancer, stomach cancer, cervical cancer, lung cancer, pancreatic cancer, kidney cancer, small cell lung carcinoma, non-small cell lung cancer, or multiple myeloma.

27. The method according to claim 26, wherein said subject is afflicted with colon carcinoma, breast cancer, cervical cancer, lung cancer, small cell lung carcinoma, or non-small cell lung cancer.

28. A method of potentiating radiotherapy cancer treatment, comprising administering to a patient in need thereof a therapeutically effective amount of a triazolone compound of 3-(2,4-dihydroxy-5-isopropyl-phenyl)-4-(1-methyl-indol-5-yl)-5-hydroxy-[1,2,4]triazole, or 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and directing radiotherapy at a prescribed dosage to a locus of cancer, wherein the cancer is selected from the group consisting of colon carcinoma, colorectal cancer, gastric cancer, hepatocellular cancer, pancreatic cancer, breast cancer, ovarian cancer, prostate cancer, stomach cancer, cervical cancer, pancreatic cancer, kidney cancer, lung cancer, small cell lung carcinoma, non-small cell lung cancer, and multiple myeloma.

29. The method according to claim 28, wherein said subject is afflicted with colon carcinoma, breast cancer, cervical cancer, lung cancer, small cell lung carcinoma, or non-small cell lung cancer.

30. A method of inhibiting the growth of a cell, said method comprising the steps of: (a) first contacting said cell with a compound of 3-(2,4-dihydroxy-5-isopropyl-phenyl)-4-(1-methyl-indol-5-yl)-5-hydroxy-[1,2,4]triazole, or 5-hydroxy-4-(5-hydroxy-4-(1-methyl-1H-indol-5-yl)-4H-1,2,4-triazol-3-yl)-2-isopropylphenyl dihydrogen phosphate, or a tautomer or a pharmaceutically acceptable salt thereof in an amount effective to sensitize said cell to ionizing radiation; and then (b) exposing said cell to a dose of ionizing radiation effective to inhibit the growth of said cell.

31. The method according to claim 24, wherein said radiation therapy comprises external beam radiotherapy or implanted radioactive sources.

32. The method according to claim 26, wherein said radiation therapy comprises external beam radiotherapy or implanted radioactive sources.

33. The method according to claim 28, wherein said radiation therapy comprises external beam radiotherapy or implanted radioactive sources.

34. The method according to claim 24, wherein the triazolone compound and said radiotherapy are administered concurrently, separately, or sequentially.

35. The method according to claim 26, wherein the triazolone compound and said radiotherapy are administered concurrently, separately, or sequentially.

36. The method according to claim 28, wherein the triazolone compound and said radiotherapy are administered concurrently, separately, or sequentially.

37. The method according to claim 24, further comprising administering one or more additional chemotherapy agents.

38. The method according to claim 26, further comprising administering one or more additional chemotherapy agents.

39. The method according to claim 28, further comprising administering one or more additional chemotherapy agents.
